(19) United States
(12) Patent Application Publication
(10) Pub. No.: US 2019/0374481 A1
(43) Pub. Date: Dec. 12, 2019

(54) ADMINISTRATION REGIMEN FOR A PHARMACEUTICAL PATCH COMPRISING LIDOCAINE AND DICLOFENAC

(71) Applicant: TEIKOKU SEIYAKU CO., LTD., Kagawa (JP)

(72) Inventors: Antonio NARDI, Herzogenrath (DE); Klaus SCHIENE, Jüchen (DE); Thomas CHRISTOPH, Aachen (DE); Sebastian WACHTEN, Aachen (DE); Irmgard BÖSL, Alsdorf (DE); Andreas SCHOLZ, Gießen (DE); Ingo FRIEDRICH, Marburg (DE); Sebastian ULLRICH, Aachen (DE); Simone KÖNIG, Aachen (DE)

(73) Assignee: TEIKOKU SEIYAKU CO., LTD., Kagawa (JP)

(21) Appl. No.: 16/481,613

(22) PCT Filed: Jan. 29, 2018
(86) PCT No.: PCT/EP2018/052033

§ 371 Date: Jul. 29, 2019

(30) Foreign Application Priority Data
Jan. 31, 2017 (EP) 17154011.5

(51) Int. Cl. A61K 9/70; A61K 31/167; A61K 31/196; A61K 47/20; A61K 47/38; A61P 25/00

(52) U.S. Cl. CPC A61K 9/7061; A61K 31/167; A61P 25/00; A61K 47/20; A61K 47/38; A61K 31/196

(57) ABSTRACT

The invention relates to a pharmaceutical patch comprising a Lidocaine constituent and a Diclofenac constituent, wherein the relative weight content ratio of the Lidocaine constituent to the Diclofenac constituent is within the range of from about 7:1 to about 4:1, based on the equivalent weight of the non-salt form of Lidocaine and of Diclofenac, for use in the local treatment or prevention of pain. The pharmaceutical composition is suitable for topical administration and local pharmacological action via delivery of Lidocaine and Diclofenac into the skin and possibly also through and to other deeper tissues such as the synovial fluid without significant systemic exposure.

The invention relates to a pharmaceutical patch comprising a Lidocaine constituent and a Diclofenac constituent, wherein the relative weight ratio of the Lidocaine constituent to the Diclofenac constituent is within the range of from about 7:1 to about 4:1, based on the equivalent weight of the non-salt form of Lidocaine and of Diclofenac, for topical use in the local treatment or prevention of pain, wherein the pharmaceutical patch is applied and remains applied to an area of the skin of a patient for an application period of more than about 12 hours, but preferably less than about 24 hours, in particular for about 18 hours. The pharmaceutical patch is suitable for locally delivering Lidocaine and Diclofenac into the skin and possibly also to other tissues such as synovial fluid without significant systemic exposure.

A wide variety of pharmaceutical preparations for topical administration of pharmacologically active ingredients is known from the prior art, such as creams, gels, ointments and the like. For the treatment of localized pain (skin pain, muscle pain or joint pain) these preparations may be topically applied onto the skin.

A more sophisticated means for topical application to the skin is a pharmaceutical patch. Most pharmaceutical patches, however, are designed for systemic, i.e. transdermal administration of pharmacologically active ingredients, but not for local administration thereof. The working principle of a pharmaceutical patch for transdermal administration relies on the release of the pharmacologically active ingredient(s) from the patch, its/their penetration into and through the skin barrier, and its/their entry into the systemic circulation through the perfused subcutaneous tissue, where it then develops its pharmacological effect at the targeted receptors. The penetration of a pharmacologically active ingredient(s) through the skin is largely determined by its/their physicochemical properties and so far, there are only relatively few preparations of pharmacologically active ingredients that are suitable for dermal administration.

Pharmaceutical patches may also be useful for non-systemic, i.e. dermal administration of pharmacologically active ingredients. In general, besides the desired pharmacological effect of pain relief, a pharmaceutical patch for dermal administration of e.g. an analgesic should satisfy the following requirements:
good adhesion to the skin without skin irritations at the contact area, even after long term application;
appropriate size that is as inconspicuous as possible;
good shelf-life and storage stability, e.g. no recrystallization of the pharmacologically active ingredient(s), reduction or even suppression of chemical degradation of the pharmacologically active ingredient(s); and
well-adjusted flux rate to make available to the relevant targeted tissues as much as possible of the pharmacologically active ingredient(s) contained in the pharmaceutical patch over a predetermined period of time at a constant or nearly constant flux rate.

Compositions for topical administration of Lidocaine as well as compositions for topical administration of Diclofenac are known from the prior art. Lidocaine patches are commercialized under the tradenames Lidoderm® and Versatis®. According to the summary of product characteristics (SmPC), when using Versatis® for treating chronic pain conditions. Versatis® has been approved for 12 h on, 12 h off per 24 h, for 1 to 3 patches used simultaneously with no limitation to long-term-use. A Diclofenac patch is commercialized under the tradename Flector Tissugel®. A gel formulation of Diclofenac is available under the tradename Voltaren®. According to the SmPC, when using Flector Tissugel®, within 24 h a first patch is to be applied for 12 h, removed and replaced by the second patch for the next 12 h, i.e. two patches are applied over 24 h without a patch-free interval. This regimen is limited to 14 days and no long-term use has been approved.

US 2005/256187 relates to a method and composition for synergistic topical therapy of the symptoms of neuromuscular pains. In this method, for intact skin or open skin, the use of a suitable topical pharmaceutical formulation is described. This formulation is loaded with a suitable concentration of a sodium channel blocker from the class of local anesthetics of the ester or amide type and a substance from the class of non-steroidal anti-inflammatory drugs, whose selective release takes place onto or under the skin region. By the simultaneous inhibition of the inflammatory pain factors at the cellular level and also of the transmission of neuronal pain impulses in reaction thereto, this therapy achieves pharmacologically more effective alleviation of neuromuscular pain.

US 2011/008413 relates to transdermal drug delivery systems. More particularly, US 2011/008413 provides compositions and transdermal drug delivery systems for the treatment and/or relief of symptoms associated with carpal tunnel syndrome or tendonitis.

US 2011/033545 relates to topical pharmaceutical preparations and methods for the treatment of acute and chronic pain and inflammation therewith.

U.S. Pat. No. 7,018,647 relates to an external skin patch having painkilling effect for pains accompanied by inflammation, such as chronic arthrorheumatism, arthrosis deformans or low back pain. The external skin patch is obtained by coating a drug-containing base on a substrate; the drug-containing base comprises an adhesive gel base containing a water soluble polymeric material, a crosslinking agent, water and a humectant as essential components, and a local anesthetic and a nonsteroidal antiphlogistic analgesic agent as medicinal components.

EP 1 405 646 discloses a salt (I) of a local anesthetic (preferably Lidocaine) with an antiinflammatory compound (II) (preferably Diclofenac).

Analgesic compositions of the prior art are not satisfactory in every respect. It is an object of the invention to provide a medicament that is useful for the local treatment or prevention of pain and that has advantages compared to the medicaments of the prior art. It would be desirable to achieve improved and prolonged pain relief as well as other benefits to the patient in terms of compliance, tolerability, and the like, especially but not limited to cases in the absence of allodynia to the skin.

This object has been achieved by the subject-matter of the patent claims, especially by a pharmaceutical patch comprising a Lidocaine constituent and a Diclofenac constituent, wherein the relative weight content ratio of the Lidocaine constituent to the Diclofenac constituent is within the range of from about 7:1 to about 4:1 respectively, based on the equivalent weight of the non-salt form of Lidocaine and of Diclofenac, for topical use in the local treatment or prevention of pain, wherein the pharmaceutical patch is applied and remains applied to an area of the skin of a patient for an application period of more than about 12 hours, but preferably less than about 24 hours, in particular for about 18 hours. In a preferred embodiment, the pain is neuropathic pain or wherein the pain has a neuropathic pain component; and/or

It has been surprisingly found that Lidocaine and Diclofenac exert a synergistic action in the treatment of pain once they are simultaneously applied topically onto the skin, hence the invention providing benefit to the patient by means of co-administration in a topical formulation, such as a pharmaceutical topical patch.

In particular, it has been surprisingly found that the pharmacological action of Diclofenac is prolonged and increased due to the topical co-administration with Lidocaine. A prolonged pharmacological action of Lidocaine can be desirable as well. A prolonged action of Diclofenac could principally imply both a pharmacokinetic and a pharmacodynamic interaction with Lidocaine. However, concomitant intravenous administration in vivo in the rat and in vitro Frantz cell experiments revealed that there is lack of pharmacokinetic interaction. Thus, it appears by surprise that the interaction of Lidocaine and Diclofenac is of pharmacological type.

The combination of Diclofenac and Lidocaine in a pharmaceutical patch according to the invention unexpectedly provides evidence of significant therapeutic superiority compared to the single chemical agents due to a pharmacological interaction. Surprisingly, the addition of increasing doses of Lidocaine bestowed both a long-term pharmacological potentiation of Diclofenac and a coalistic pharmacological effect (neither drug active individually) across the diverse experimental testing. In view of these experimental findings, a pharmaceutical patch has been designed comprising Diclofenac and Lidocaine with a molar excess of Lidocaine in order to replicate and to fully exploit in clinical settings the pharmacologically potentiating and coalistic effects of Diclofenac in presence of a higher molar ratio of Lidocaine. The duration of the pharmacological effect of Diclofenac is expected to be a function of the amount of Lidocaine in the patch; the more Lidocaine, the stronger and/or longer the pharmacological effect of Diclofenac.

Further, it has been surprisingly found that when applying the combination of Diclofenac and Lidocaine according to the invention to the skin, local relief of neuropathic pain can be unexpectedly achieved. Diclofenac alone is regarded as pharmacologically ineffective against neuropathic pain. Lidocaine alone has analgesic action against neuropathic pain that is localized superficially in the skin (allodynia) but not when located in anatomically deeper structures. However, when being administered topically/locally, a Lidocaine patch alone has no analgesic action against nociceptive pain. It has been surprisingly found that when topically/locally co-administering Diclofenac and Lidocaine, analgesic action can be achieved against neuropathic pain and against pain having a neuropathic component. The topical co-administration of Lidocaine and Diclofenac appears to promote the alleviation of neuropathic pain due to the inhibition of the previously un-accessible inflammatory component. The mutual interactions of nociceptive pain with neuropathic pain are currently neither measurable/quantifiable nor predictable. Nociceptive pain may turn into neuropathic pain leading to so-called mixed pain situations involving both pain components, a nociceptive pain component as well as a neuropathic pain component.

Furthermore, it has been surprisingly found that the combination of Diclofenac and Lidocaine according to the invention is particularly useful for treating those patients affected by renal and/or hepatic impairment. In view of the newly-discovered retention or potentiation of the therapeutic benefit by topical/local administration of the drug combination and the resulting negligible systemic plasma concentration of both drugs, patients that otherwise could not be treated systemically with Diclofenac and/or Lidocaine due to renal and/or hepatic impairment, may now be treated with the pharmaceutical patch according to the invention.

When the pharmaceutical patch according to the invention is topically applied to the skin, Lidocaine and Diclofenac are administered dermally, i.e. penetrate into the skin (intradermally) possibly also through the skin into subcutaneous regions like subcutaneous tissues, muscles, synovial fluid, and the like in a sufficient amount and rate to elicit the desired analgesic effect locally.

As it has been unexpectedly found that Lidocaine and Diclofenac may act synergistically, thus allowing for the treatment of pain conditions that so far have not been treated with Lidocaine and/or Diclofenac singularly, especially diseases or conditions characterized by inflammatory pain and neuropathic pain. Examples of inflammatory pain include but are not limited to pain due to osteoarthritis and low-back pain.

A first aspect of the invention relates to a pharmaceutical patch comprising a Lidocaine constituent and a Diclofenac constituent, wherein the relative weight ratio of the Lidocaine constituent to the Diclofenac constituent is within the range of from about 7:1 to about 4:1, preferably from about 6.5:1 to about 4.5:1, more preferably from about 6:1 to about 5:1, in each case based on the equivalent weight of the non-salt form of Lidocaine and of Diclofenac, for topical use in the local treatment or prevention of pain, wherein the pharmaceutical patch is applied and remains applied to an area of the skin of a patient for an application period of more than about 12 hours.

Preferably, the pain is neuropathic pain, or wherein the pain has a neuropathic pain component e.g. inflammatory pain having a neuropathic component, or wherein the pain is of unclear quantitative origin of either nociceptive pain and/or neuropathic pain due to a mixed-pain situation.

A skilled person recognizes that a pharmaceutical patch is usually composed of various elements. While some elements of a pharmaceutical patch are usually capable of and devoted for housing or comprising a pharmaceutical composition such as an adhesive layer (drug in the adhesive layer) or a separate depot or reservoir layer, other elements of a pharmaceutical patch are typically neither devoted for nor capable of housing or comprising a pharmaceutical composition such as a backing layer or a release liner.

For the purpose of the specification, unless expressly stated otherwise, all weight percentages relate to the total weight of the pharmaceutical patch or to the total weight of a specific layer thereof in terms of total per dry unit. In this regard, “dry unit” shall encompass all constituents, irrespective of whether they are present in solid, semisolid or liquid form, but shall not encompass volatile solvents that are evaporated in course of the preparation of the pharmaceutical patch such as ethanol, heptane, ethyl acetate and the like. Thus, “dry unit” shall merely encompass the residual content of volatile solvent(s), e.g. water of a hydrogel, if any.

The pharmaceutical patch according to the invention preferably comprises a surface layer, an adhesive layer, and a removable protective layer, wherein the adhesive layer is preferably located between the surface layer (also referred to as “backing layer”) and the removable protective layer (also referred to as “release liner”).

The Lidocaine constituent and the Diclofenac constituent may be contained in the same layer(s) of the pharmaceutical patch or at least partially in different layers.

In a preferred embodiment, the adhesive layer comprises at least a portion of the total amount of the Lidocaine constituent and at least a portion of the total amount of the Diclofenac constituent that is contained in the pharmaceutical patch.

In a preferred embodiment, the adhesive layer is adjacent to the removable protective layer and/or to the surface layer. Preferably, the adhesive layer is adjacent to the removable protective layer and to the surface layer. In a particularly preferred embodiment, the pharmaceutical patch is composed of the surface layer, the adhesive layer, and the removable protective layer and does not contain any additional layer.

Preferably, the Lidocaine constituent and the Diclofenac constituent are contained in an adhesive layer (matrix patch) of the pharmaceutical patch according to the invention. A matrix patch contains the pharmacologically active ingredient in a matrix that typically is also the adhesive layer that provides adhesion of the pharmaceutical patch to the skin (drug in adhesive).

The adhesive layer is preferably located between the surface layer and the removable protective layer. Preferably, the surface layer forms the outer surface of the pharmaceutical patch, i.e. when the pharmaceutical patch is applied to the skin, the surface layer is the visible layer of the pharmaceutical patch.

Preferably, one of the two opposing surfaces of the adhesive layer is in intimate contact with, i.e. adjacent to the removable protective layer.

In a preferred embodiment, the other of the two opposing surfaces of the adhesive layer is in intimate contact with the surface layer, which in turn preferably forms on its outer surface the outer surface of the pharmaceutical patch. According to this embodiment of the invention, the pharmaceutical patch preferably consists of surface layer, adhesive layer and removable protective layer, so that the adhesive layer contains the Lidocaine constituent and the Diclofenac constituent (drug-in-adhesive).

The adhesive layer containing the Lidocaine constituent and the Diclofenac constituent may be present in form of a liquid, a semisolid, or a solid polymer matrix.

In a preferred embodiment, the adhesive layer comprises a liquid, preferably water, containing the Lidocaine constituent and the Diclofenac constituent in form of a solution or suspension.

In another preferred embodiment, the adhesive layer is a semisolid, such as a gel, or a solid polymer matrix wherein the Lidocaine constituent and/or the Diclofenac constituent is dispersed, preferably dissolved.

In a preferred embodiment, the total amount of the Lidocaine constituent and/or Diclofenac constituent is present in molecular dispersed form.

In another preferred embodiment, only a portion of the Lidocaine constituent and/or only a portion of the Diclofenac constituent is present in molecular dispersed form, while the remainder of the Lidocaine constituent and/or the remainder of the Diclofenac constituent is present is non-molecular dispersed form (e.g. in form of droplets, crystals and the like) serving the purpose of a depot, also called “microreservoir”.

Preferably, the Lidocaine constituent and/or Diclofenac constituent is contained in the adhesive layer, while a certain portion of the Lidocaine constituent and/or Diclofenac constituent may be contained in the adjacent layers e.g. due to migration and/or diffusion.

In a preferred embodiment, the adhesive layer is a hydrogel comprising at least a portion of the Lidocaine constituent and at least a portion of the Diclofenac constituent, preferably essentially the total amount of the Lidocaine constituent and essentially the total amount of the Diclofenac constituent.

After being applied to the skin, preferably to human skin, the pharmaceutical patch according to the invention preferably releases the Lidocaine constituent as well as the Diclofenac constituent independently of one another in the form as they are contained in the pharmaceutical composition or in a modified form thereof. For example, when the Lidocaine constituent is Lidocaine in non-salt form, the pharmaceutical patch according to the invention preferably releases Lidocaine in non-salt form. When the Diclofenac constituent is Diclofenac in salt form, e.g. Diclofenac potassium salt, the pharmaceutical patch according to the invention preferably either releases Diclofenac in non-salt form or in salt form.

In a preferred embodiment, the pharmaceutical patch according to the invention is for local administration of the Lidocaine constituent and/or the Diclofenac constituent.

Preferably, the routes of administration for the Lidocaine constituent and for the Diclofenac constituent are identical, i.e. preferably the Lidocaine constituent and the Diclofenac constituent are both for local administration, or the Lidocaine constituent and the Diclofenac constituent are both for parenteral local administration. However, the invention also encompasses embodiments where the Diclofenac constituent is for local administration whereas the Lidocaine constituent is for parenteral local administration and embodiments where the Lidocaine constituent is for local administration whereas the Diclofenac constituent is for parenteral local administration.

Preferably, the pharmaceutical patch has an area of at least 5 cm², at least 10 cm² or at least 20 cm², more preferably at least 30 cm², at least 40 cm² or at least 50 cm², still more preferably at least 60 cm², at least 70 cm² or at least 80 cm², and most preferably at least 90 cm², at least 100 cm² or at least 110 cm². Preferably, the skin contact surface within the range of from about 50 cm² to about 250 cm², more preferably from about 100 cm² to about 200 cm².

The pharmaceutical patch according to the invention may have various shapes, e.g. round or rectangular, the latter with or without rounded corners. When the pharmaceutical patch is to be applied to the skin of a joint, e.g. knee or elbow, its shape may be customized so as to allow patient's unimpeded movement.

The total thickness of the pharmaceutical patch according to the invention is not particularly limited.

Preferably, the total thickness of the pharmaceutical patch is within the range of from 500 μm to 2500 μm, more preferably 750 μm to 2000 μm, still more preferably 1000 μm to 1750 μm.

The pharmaceutical patch according to the invention comprises a Lidocaine constituent, i.e. Lidocaine or a physiologically acceptable salt thereof.

Lidocaine (i.e. 2-(diethylamino)-N-(2,6-dimethylphenyl)acetamide), also known as xylocaine and lignocaine, is useful to numb tissue in a specific area, to treat ventricular tachycardia, and for nerve blocks. Lidocaine has the following structure:

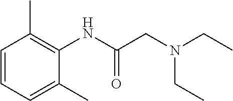 [SMILES: CCN(CC)CC(=O)Nc1c(C)cccc1C]

Lidocaine is an antiarrhythmic medication of the class Ib type. Lidocaine works by blocking sodium channels and thus decreasing the rate of contractions of the heart. When used locally as a numbing agent, local neurons cannot signal to the brain.

For the purpose of the specification, the expression “Lidocaine constituent” refers to Lidocaine in a non-salt form or to Lidocaine in form of a physiologically-acceptable salt. Furthermore, the expression “Lidocaine constituent” also encompasses any solid form such as polymorphs, any aggregate with other molecules such as solvates, co-crystals, and the like, as well as any derivative such as prodrugs.

Preferably, the Lidocaine constituent comprises, essentially consists of or is Lidocaine or a physiologically acceptable salt thereof, preferably the non-salt form of Lidocaine. It is also possible that Lidocaine forms a salt with Diclofenac. Unless expressly stated otherwise, all values defining the content or the quantity of the Lidocaine constituent in accordance with the invention are based on the equivalent weight of the non-salt form of Lidocaine.

The content of the Lidocaine constituent in the pharmaceutical patch according to the invention is not particularly limited.

Preferably, the concentration (content) of the Lidocaine constituent in the adhesive layer is at least 0.10 wt.-%, more preferably at least 0.20 wt.-%, still more preferably at least 0.30 wt.-%, yet more preferably at least 0.40 wt.-%, even more preferably at least 0.50 wt.-%, most preferably at least 0.60 wt.-% and in particular at least 0.70 wt.-%, relative to the total weight of the adhesive layer and based on the weight of the non-salt form of Lidocaine.

Preferably, the content of the Lidocaine constituent is within the range of from 0.01 wt.-% to 50 wt.-%, more preferably 0.1 wt.-% to 10 wt.-%, relative to the total weight of the adhesive layer and based on the weight of the non-salt form of Lidocaine.

Preferably, the content of the Lidocaine constituent in the adhesive layer is within the range of from about 2.5 wt.-% to about 7.5 wt.-%, more preferably from about 3.0 wt.-% to about 7.0 wt.-%, still more preferably from about 3.5 wt.-% to about 6.5 wt.-%, yet more preferably from about 4.0 wt.-% to about 6.0 wt.-%, most preferably from about 4.5 wt.-% to about 5.5 wt.-%, in each case based on the weight of the non-salt form of Lidocaine.

Preferably, the area concentration of the Lidocaine constituent in the adhesive layer is within the range of from 0.01 to 100 g/m². A typical area concentration is e.g. 700 mg in 140 cm², i.e. 0.7 g/0.014 m²=50 g/m² (5 mg/cm²). Preferably, the area concentration of the Lidocaine constituent in the adhesive layer is within the range of 5.0±4.0 mg/cm², more preferably 5.0±3.0 mg/cm², still more preferably 5.0±2.0 mg/cm², and most preferably 5.0±1.0 mg/cm², in each case based on the weight of the non-salt form of Lidocaine.

The total dose of the Lidocaine constituent and/or Diclofenac constituent that is contained in the pharmaceutical patch is not particularly limited and may depend upon various factors such as body weight of the subject to be treated and duration of application on the skin.

Preferably, the total dose of the Lidocaine constituent is within the range of from about 600 mg to about 800 mg, more preferably from about 650 mg to about 750 mg, most preferably about 700 mg (e.g. 699 mg or 701 mg), in each case based on the weight of the non-salt form of Lidocaine.

The pharmaceutical patch according to the invention comprises a Diclofenac constituent, i.e. Diclofenac or a physiologically acceptable salt thereof.

Diclofenac is a nonsteroidal anti-inflammatory drug (NSAID) taken or applied to reduce inflammation and as an analgesic reducing pain in certain conditions. The name “diclofenac” derives from its chemical name: 2-(2,6-dichloranilino) phenylacetic acid:

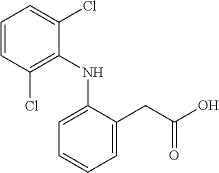 [SMILES: O=C(O)Cc1ccccc1Nc1c(Cl)cccc1Cl]

For the purpose of the specification, the expression “Diclofenac constituent” refers to Diclofenac in non-salt form or to Diclofenac in form of a physiologically-acceptable salt, especially the sodium salt, most preferably the epolamine salt. Furthermore, the expression “Diclofenac constituent” also encompasses any solid form such as polymorphs, any aggregate with other molecules such as solvates, co-crystals, and the like, as well as any derivative such as prodrugs.

Preferably, the Diclofenac constituent comprises, essentially consists of or is Diclofenac or a physiologically-acceptable salt thereof, preferably Diclofenac epolamine. It is also possible that Diclofenac forms a salt with Lidocaine. Unless expressly stated otherwise, all values defining the content or the quantity of the Diclofenac constituent in accordance with the invention are based on the equivalent weight of the non-salt form of Diclofenac.

The content of the Diclofenac constituent in the pharmaceutical patch according to the invention is not particularly limited.

Preferably, the concentration (content) of the Diclofenac constituent in the adhesive layer is at least 0.10 wt.-%, more preferably at least 0.20 wt.-%, still more preferably at least 0.30 wt.-%, yet more preferably at least 0.40 wt.-%, even more preferably at least 0.50 wt.-%, most preferably at least 0.60 wt.-% and in particular at least 0.70 wt.-%, relative to the total weight of the adhesive layer and based on the weight of the non-salt form of Diclofenac.

Preferably, the content of the Diclofenac constituent is within the range of from 0.01 wt.-% to 50 wt.-%, more preferably 0.1 wt.-% to 10 wt.-%, relative to the total weight of the adhesive layer and based on the weight of the non-salt form of Diclofenac.

Preferably, the content of the Diclofenac constituent in the adhesive layer is within the range of from about 0.50 wt.-% to about 1.40 wt.-%, more preferably from about 0.60 wt.-% to about 1.30 wt.-%, still more preferably from about 0.70 wt.-% to about 1.20 wt.-%, yet more preferably from about 0.80 wt.-% to about 1.10 wt.-%, most preferably from about 0.90 wt.-% to about 1.00 wt.-%, in each case based on the weight of the non-salt form of Diclofenac.

Preferably, the area concentration of the Diclofenac constituent in the adhesive layer and the drug layer, respectively, is within the range of from 0.01 to 50 g/m². A typical area concentration is e.g. 130 mg in 140 cm², i.e. 0.13 g/0.014 m²=9.3 g/m² (0.93 mg/cm²). Preferably, the area concentration of the Diclofenac constituent in the adhesive layer is within the range of 0.93±0.80 mg/cm², more preferably 0.93±0.60 mg/cm², still more preferably 0.93±0.40 mg/cm², and most preferably 0.93±0.20 mg/cm², in each case based on the weight of the non-salt form of Diclofenac.

The total dose of the Diclofenac constituent that is contained in the pharmaceutical patch is not particularly limited and may depend upon various factors such as body weight of the subject to be treated and duration of application on the skin.

Preferably, the total dose of the Diclofenac constituent is within the range of from about 85 mg to about 200 mg, more preferably from about 100 mg to about 150 mg, most preferably about 130 mg (e.g. 129 mg or 131 mg), based on the weight of the non-salt form of Diclofenac.

The relative weight ratio of the Lidocaine constituent to the Diclofenac constituent is within the range of from about 7:1 to 5:1, preferably from about 6.5:1 to about 4.5:1, more preferably from about 6:1 to about 5:1, in each case based on the equivalent weight of the non-salt form of Lidocaine and of Diclofenac.

Lidocaine in its non-salt form has a molecular weight of about 234 g/mol. Diclofenac in its non-salt form has a molecular weight of about 296 g/mol. Thus, a relative weight ratio of the Lidocaine constituent to the Diclofenac constituent of 7:1, both expressed in terms of the equivalent weight of the non-salt form of Lidocaine and the non-salt-form of Diclofenac, corresponds to a relative molar ratio of about 8.85:1, whereas a relative weight ratio of 5:1 corresponds to a relative molar ratio of about 6.32:1.

Preferably, the relative weight ratio of the Lidocaine constituent to the Diclofenac constituent is such that the composition provides a synergistic effect with respect to pain relief.

The pharmaceutical patch according to the invention preferably comprises an adhesive layer.

Preferably, the adhesive layer comprises a polymer that forms a matrix in which the Lidocaine constituent and/or Diclofenac constituent is dispersed (drug-in-adhesive).

Preferably, the adhesive layer comprises a pressure sensitive adhesive selected from the group consisting of

(i) polysilicone based pressure sensitive adhesives,
(ii) polyacrylate based pressure sensitive adhesives,
(iii) polyisobutylene based pressure sensitive adhesives,
(iv) styrenic rubber based pressure sensitive adhesives, and
(v) hydrogel based pressure sensitive adhesives.

The thickness of the adhesive layer is not particularly limited and may depend upon a number of factors such as function within the patch (e.g. drug-in-adhesive), content of Lidocaine constituent and/or Diclofenac constituent and excipients, prescribed duration of application of pharmaceutical patch on the skin, and the like.

Preferably, the adhesive layer has a thickness within the range of from 1.0 to 1000 μm.

The ratio of the thickness of the surface layer to the thickness of the adhesive layer is not particularly limited. In a preferred embodiment, the thickness of the surface layer is greater than the thickness of the adhesive layer. In another preferred embodiment, the thickness of the adhesive layer is greater than the thickness of the surface layer.

Preferably, the pressure sensitive adhesive contained in the adhesive layer comprises a hydrogel that is preferably based on a hydrogel former, a hydrocolloid, a hydrophilic gel former, or a mixture thereof.

Preferred hydrophilic gel formers, hydrogel formers, and hydrocolloids, respectively, include but are not limited to inorganic hydrogel formers, natural organic hydrogel formers, semi-synthetic organic hydrogel formers and synthetic organic hydrogel formers. Preferred natural organic hydrogel formers include agar, alginic acid and alginates, arabic gum, gelatin, tragacanth and starches such as potato starch, corn starch, rice starch and wheat starch. Preferred semi-synthetic organic hydrogel formers include cellulose derivatives, preferably methyl cellulose, hydroxyethyl cellulose, carmellose sodium and hypromellose (hydroxypropyl methylcellulose). Preferred synthetic organic hydrogel former include polyacrylic acids, polyacrylates, polyvinyl alcohols, povidones and copovidones.

A hydrogel may for example be based on a polymeric mixture comprising poly(vinyl alcohol), carmellose-sodium, poly(acrylic acid), poly(acrylic acid sodium salt), and gelatine.

Additional constituents of the hydrogel may include but are not limited to solvents such as glycerol, propylenglycol, and/or purified water; humectants such as sorbitol and/or urea.

The adhesive layer of the pharmaceutical patch may contain other pharmaceutical excipients that are conventionally contained in pharmaceutical patches.

Preferably, the adhesive layer of the pharmaceutical patch comprises a crystallization inhibitor which inhibits the crystallization of the Lidocaine constituent and/or Diclofenac constituent within the adhesive layer and drug layer, respectively. Thus, the crystallization inhibitor is preferably contained in the same layer as the Lidocaine constituent and/or Diclofenac constituent. Preferably, the content of the crystallization inhibitor within said layer is within the range of from 1.0 to 20 wt.-%, more preferably 2.5 to 17.5 wt.-%, still more preferably 5.0 to 15 wt.-%, yet more preferably 6.0 to 14 wt.-%, even more preferably 7.0 to 13 wt.-%, most preferably 8.0 to 12 wt.-%, and in particular 9.0 to 11 wt.-%, relative to the total weight of said layer. Preferred crystallization inhibitors include but are not limited to polyvinylpyrrolidones (povidone, polyvidone) (e.g. Kollidon 25), N-vinyl-1-aza-cycloheptan-2-one homopolymers, N-vinylpiperidin-2-one homopolymers, polyethylene glycol, poloxamer (e.g. Lutrol F127), and copovidone (e.g. Kollidon VA64).

Preferably, the molar ratio of the total content of the Lidocaine constituent and the Diclofenac constituent to the crystallization inhibitor is within the range of from 1000:1 to 1:1000, more preferably 250:1 to 1:250, still more preferably 100:1 to 1:100, yet more preferably 50:1 to 1:50, even more preferably 25:1 to 1:25, most preferably 10:1 to 1:10, and in particular 5:1 to 1:5.

Preferably, the adhesive layer of the pharmaceutical patch according to the invention preferably contains a permeation constituent which enhances intradermal penetration and permeation of the Lidocaine constituent and/or Diclofenac constituent into human skin and possibly also through human skin into the tissue below, i.e. one or more intradermal penetration enhancers. Intradermal penetration enhancers are known to the skilled person (cf., e.g., Smith et al., Intradermal Penetration Enhancers, CRC Press, 1995).

Preferably, the adhesive layer of the pharmaceutical patch which contains the Lidocaine constituent and/or Diclofenac constituent contains at least one intradermal penetration enhancer.

Preferably, the relative weight ratio of the total content of the Lidocaine constituent and the Diclofenac constituent to the permeation component is within the range of from 25:1 to 1:1000, more preferably 10:1 to 1:250, still more preferably 5:1 to 1:100, yet more preferably 1:1 to 1:50, even more preferably 1:2 to 1:25, most preferably 1:6 to 1:20, and in particular 1:9 to 1:17.

Preferably, the content of the permeation component within the adhesive layer is within the range of from 1.0 to 20 wt.-%, more preferably 2.5 to 17.5 wt.-%, still more preferably 5.0 to 15 wt.-%, yet more preferably 6.0 to 14 wt.-%, even more preferably 7.0 to 13 wt.-%, most preferably 8.0 to 12 wt.-%, and in particular 9.0 to 11 wt.-%, relative to the total weight of the adhesive layer.

Preferred intradermal penetration enhancers include but are not limited to:
a) sulfoxides such as dimethylsulfoxide (DMSO) and decylmethylsulfoxide;
b) ethers such as diethylene glycol monoethyl ether (transcutol) and diethylene glycol monomethyl ether;
c) surfactants such as sodium laurate, sodium lauryl sulfate, cetyltrimethylammonium bromide, benzalkonium chloride, poloxamers, polysorbates (e.g. polysorbate 80) and lecithin;
d) 1-substituted azacycloheptan-2-ones such as 1-n-dodecylcyclazacycloheptan-2-one;
e) alcohols and fatty alcohols such as ethanol, propanol, octanol, dodecanol, oleyl alcohol, benzyl alcohol, and the like;
f) polyols, esters of polyols and ethers of polyols such as propylene glycol, ethylene glycol, diethylene glycol, dipropylene glycol, glycerol, sorbitol, butanediol, polyethylene glycol, polyvinyl alcohol (e.g. Mowiol 4-88), triacetine and polyethylene glycol monolaurate;
g) organic acids such as salicylic acid and salicylates, citric acid, levulinic acid, caprylic acid and succinic acid; as well as dicarboxylic acids and their esters such as dibutylene sebacate;
h) fatty acids such as lauric acid, oleic acid and valeric acid; fatty acid esters such as isopropyl palmitate, methylpropionate, propylene glycol monolaureate, lauryl lactate, oleyl oleate and ethyl oleate;
i) amides and other nitrogenous compounds such as urea, dimethylacetamide, dimethylformamide, 2-pyrrolidone, 1-methyl-2-pyrrolidone, ethanolamine, diethanolamine, triethanolamine and laurocapram (Azone®);
j) terpenes;
k) alkanones;
l) other oligomers or polymers;
   and mixtures of any of the foregoing.

Preferably, the adhesive layer of the pharmaceutical patch comprises an antioxidant. Suitable antioxidants include but are not limited to alpha-tocopherol, butyl hydroxytoluene, ascorbic acid or n-propylgalat.

Preferably, the adhesive layer comprises a chelating agent such as sodium edetate or disodium edetate.

Preferably, the adhesive layer comprises a preservative such as methyl-4-hydroxybenzoate and/or propyl-4-hydroxybenzoate.

Preferably, the content of the antioxidant is within the range of from 0.01 to 10 wt.-%, more preferably 0.05 to 7.5 wt.-%, still more preferably 0.1 to 2.5 wt.-%, yet more preferably 0.5 to 1.5 wt.-%, even more preferably 0.7 to 1.3 wt.-%, most preferably 0.8 to 1.2 wt.-%, and in particular 0.9 to 1.1 wt.-%, relative to the total weight of the adhesive layer.

Preferably, the content of the chelating agent is within the range of from 0.001 to 1.0 wt.-%, more preferably 0.005 to 0.75 wt.-%, still more preferably 0.01 to 0.25 wt.-%, yet more preferably 0.05 to 0.15 wt.-%, even more preferably 0.07 to 0.13 wt.-%, most preferably 0.08 to 0.12 wt.-%, and in particular 0.09 to 0.11 wt.-%, relative to the total weight of the adhesive layer.

Preferably, the content of the preservative is within the range of from 0.001 to 1.0 wt.-%, more preferably 0.005 to 0.75 wt.-%, still more preferably 0.01 to 0.5 wt.-%, yet more preferably 0.02 to 0.25 wt.-%, even more preferably 0.03 to 0.2 wt.-%, most preferably 0.04 to 0.15 wt.-%, and in particular 0.05 to 0.1 wt.-%, relative to the total weight of the adhesive layer.

In preferred embodiments, the pharmaceutical patch according to the invention exhibits satisfactory storage stability and shelf-life. In this regard “satisfactory” preferably means that the pharmaceutical patch according to the invention preferably satisfies the requirements for storage stability and shelf-life according to FDA and/or EMA.

The pharmaceutical patch according to the invention preferably comprises a surface layer.

The term “surface layer” as used herein refers to any layer that represents the surface layer after the application of the pharmaceutical patch. This definition includes permanent backing layer commonly used for pharmaceutical patches as well as thin non-removable films that are typically used in thin flexible patches. The surface layer may include tissues, woven materials, non-woven materials, fleece and the like. Non-woven polyethylene terephthalate is preferred.

The thickness of the surface layer is not particularly limited. Preferably, the surface layer has a thickness within the range of from 0.1 to 5000 μm.

The pharmaceutical patch according to the invention preferably comprises a removable protective layer (release liner).

Preferably, the removable protective layer comprises a polymer film and a silicone coating or fluoropolymer coating. Preferably, the polymer film is a polyolefin, in particular polyethylene or polypropylene film or polyester, in particular polyethylene terephthalate film.

In a preferred embodiment, the removable protective layer is a silicone coated polyolefin or silicone coated polyester film, such as a silicone coated polyethylene terephthalate, polypropylene or polyethylene film.

In another preferred embodiment, the removable protective layer is a fluoropolymer coated polyolefin or polyester film, such as a fluoropolymer coated polyethylene terephthalate, polypropylene or polyethylene film.

The thickness of the removable protective layer is not particularly limited. Preferably, the removable protective layer has a thickness within the range of from 0.1 to 500 μm.

The pharmaceutical patch according to the invention may be prepared by standard techniques for the manufacture of pharmaceutical patches. Such standard techniques are known to the skilled person (cf., e.g., H. A. E. Benson et al., Topical and Dermal Drug Delivery: Principles and Practice, John Wiley & Sons; 2011; A. K. Banga, Dermal and Intradermal Delivery of Therapeutic Agents: Application of Physical Technologies, CRC Press Inc; 2011).

The pharmaceutical patch according to the invention is for use in the treatment or prevention of pain, preferably moderate to severe pain. In analogy, the present invention also relates to the use of a Lidocaine constituent and/or a Diclofenac constituent according to the invention for the manufacture of a pharmaceutical patch according to the invention for the treatment or prevention of pain, preferably moderate to severe pain. In analogy, the present invention also relates to a method of treating or preventing pain, preferably moderate to severe pain, comprising the application of a pharmaceutical patch according to the invention to the skin of a subject in need thereof.

The pain to be treated or to be prevented is preferably moderate, moderate to severe, or severe. The pain may be chronic or acute; and/or central and/or peripheral; and/or neuropathic and/or nociceptive. In connection with central/peripheral pain and with nociceptive/neuropathic pain “and/or” reflects the possibility that the overall pain may have different components, e.g. a nociceptive component as well as a neuropathic component. Preferably, the pain is chronic neuropathic pain, which may be peripheral or central; acute neuropathic pain, which may be peripheral or central; chronic nociceptive pain, which may be peripheral or central; or acute nociceptive pain, which may be peripheral or central. Methods to diagnose and distinguish between different components are known to the skilled person. For example, a neuropathic pain component may be diagnosed by means of the painDETECT questionnaire.

The pain may be acute or chronic, central or peripheral, visceral, inflammatory or neuropathic.

In a preferred embodiment, the pain is acute pain or chronic pain.

In a preferred embodiment, the pain is back pain, preferably low back pain; pain due to osteoarthritis, preferably due to osteoarthritis of the knee, osteoarthritis of the hip, osteoarthritis of the hand, osteoarthritis of the spine, or osteoarthritis of the elbow; visceral pain, rheumatoid pain, musculoskeletal pain, joint pain, gout pain, or inflammatory pain.

Preferably, the pharmaceutical composition as well as the pharmaceutical patch according to the invention is suitable for use in the treatment or prevention of inflammatory pain, preferably chronic arthritic pain and low back pain, also with neuropathic pain component.

For the purpose of the specification, neuropathic pain is pain that originates from nerve damage or nerve malfunction. Preferably, the neuropathic pain is selected from acute neuropathic pain and chronic neuropathic pain. Neuropathic pain may be caused by damage or disease affecting the central or peripheral portions of the nervous system involved in bodily feelings (the somatosensory system). Preferably, the pharmaceutical patch according to the invention is for use in the treatment of chronic neuropathic pain or acute neuropathic pain, peripheral neuropathic pain or central neuropathic pain, mononeuropathic pain or polyneuropathic pain. When the neuropathic pain is chronic, it may be chronic peripheral neuropathic pain or chronic central neuropathic pain, in a preferred embodiment chronic peripheral mononeuropathic pain or chronic central mononeuropathic pain, in another preferred embodiment chronic peripheral polyneuropathic pain or chronic central polyneuropathic pain. When the neuropathic pain is acute, it may be acute peripheral neuropathic pain or acute central neuropathic pain, in a preferred embodiment acute peripheral mononeuropathic pain or acute central mononeuropathic pain, in another preferred embodiment acute peripheral polyneuropathic pain or acute central polyneuropathic pain.

Central neuropathic pain is found in spinal cord injury, multiple sclerosis, and some strokes. Fibromyalgia is potentially a central pain disorder and is responsive to medications that are effective for neuropathic pain. Accordingly, the pharmaceutical patch according to the invention is also suitable for the treatment of fibromyalgia, complex regional pain syndrome (CRPS) and erythromelalgia (Mitchell's disease). Aside from diabetic neuropathy and other metabolic conditions, the common causes of painful peripheral neuropathies are herpes zoster infection, HIV-related neuropathies, nutritional deficiencies, toxins, remote manifestations of malignancies, genetic, and immune mediated disorders or physical trauma to a nerve trunk. Neuropathic pain is common in cancer as a direct result of cancer on peripheral nerves (e.g., compression by a tumor), or as a side effect of chemotherapy, radiation injury or surgery.

In a particularly preferred embodiment of the invention, the pain is neuropathic pain or pain having a neuropathic component.

In preferred embodiments of the invention, the neuropathic pain or the pain having a neuropathic component is selected from the group consisting of trigeminal neuralgia [G50.0], radiculopathy [M54.1], cervicalgia [M54.2], sciatica [M54.3], dorsalgia [M54.8, M54.9], myalgia [M79.1], fibromyalgia, paraesthesia of skin [R20.2], diabetic neuropathy, chemically induced neuropathy, shingles, HW-induced neuropathy, and neuropathy from injury, wherein the information in brackets refers to the classification according to ICD-10.

In preferred embodiments of the invention, the neuropathic pain or the pain having a neuropathic component is selected from the group consisting of low back pain, pain due to osteoarthritis preferably osteoarthritis of the knee, osteoarthritis of the hip, osteoarthritis of the hand, osteoarthritis of the spine, or osteoarthritis of the elbow; pain being or being associated with panic disorder [episodic paroxysmal anxiety] [F41.0], persistent somatoform pain disorder [F45.4], pain disorders exclusively related to psychological factors [F45.41]; nonorganic dyspareunia [F52.6]; migraine [G43]; other headache syndromes [G44]; atypical facial pain [G50.1]; phantom limb syndrome with pain [G54.6]; acute and chronic pain, not elsewhere classified [G89]; ocular pain [H57.1]; shoulder pain [M25.51]; spine pain [M54.]; low back pain [M54.5]; pain in thoracic spine [M54.6]; other shoulder lesions [M75.8]; other soft tissue disorders, not elsewhere classified [M79]; neuralgia and neuritis, unspecified [M79.2]; pain in limb [M79.6]; other specified disorders of bone [M89.8]; pain localized to upper abdomen [R10.1]; headache [R51]; pain, not elsewhere classified [R52]; chronic intractable pain [R52.1]; other chronic pain [R52.2]; pain, unspecified [R52.9]; dengue with warning signs [A97.1]; neurasthenia [F48.0]; other specified neurotic disorders [F48.8]; neurotic disorder, unspecified [F48.9]; dyspareunia [N94.1]; vaginismus [N94.2]; wherein the information in brackets refers to the classification according to ICD-10.

Nociceptive pain refers to the discomfort that results when a stimulus causes tissue damage to the muscles, bones, skin or internal organs. For the purpose of the specification, nociceptive pain is caused by stimulation of peripheral nerve fibers that respond only to stimuli approaching or exceeding harmful intensity (nociceptors), and may be classified according to the mode of noxious stimulation; the most common categories being “thermal” (heat or cold), “mechanical” (crushing, tearing, etc.) and “chemical” (iodine in a cut, chili powder in the eyes). Nociceptive pain may also be divided into “visceral,” “deep somatic” and “superficial somatic” pain.

Visceral pain describes a type of nociceptive pain originating in the body's internal organs or their surrounding tissues. This form of pain usually results from the infiltration of harmful cells, as well as the compression or extension of healthy cells. Patients suffering from visceral pain tend to feel generally achy, as this pain tends to not be localized to a specific area. Cancer is a common source of visceral pain. Examples of visceral pain include but are not limited to angina pectoris, unspecified [120.9]; other specified diseases of anus and rectum [K62.8]; unspecified renal colic [N23]; other specified disorders of penis [N48.8]; other specified disorders of male genital organs [N50.8]; mastodynia [N64.4]; pain and other conditions associated with female genital organs and menstrual cycle [N94]; mittelschmerz [N94.0]; other specified conditions associated with female genital organs and menstrual cycle [N94.8]; abdominal and pelvic pain [R10]; pelvic and perineal pain [R10.2]; pain localized to other parts of lower abdomen [R10.3]; other and unspecified abdominal pain [R10.4]; pain associated with micturition [R30]; other and unspecified symptoms and signs involving the urinary system [R39.8]; nonorganic vaginismus [F52.5]; nonorganic dyspareunia [F52.6]; painful micturition, unspecified [R30.9]; wherein the information in brackets refers to the classification according to ICD-10.

Somatic pain is nociceptive pain that results from some injury to the body. It's generally localized to the affected area and abates when the body repairs the damage to that area. Deep somatic pain is initiated by stimulation of nociceptors in ligaments, tendons, bones, blood vessels, fasciae and muscles, and is dull, aching, poorly-localized pain. Examples include sprains and broken bones. Superficial pain is initiated by activation of nociceptors in the skin or superficial tissues, and is sharp, well-defined and clearly located.

According to the invention, nociceptive pain is preferably classified chronic if it has occurred for at least 3 months. Preferably, the chronic nociceptive pain is selected from chronic visceral pain, chronic deep somatic pain and chronic superficial somatic pain.

In preferred embodiments, the pain is selected from the group consisting of otalgia [H92.0]; other specified disorders of nose and nasal sinuses [J34.8]; other diseases of pharynx [J39.2]; temporomandibular joint disorders [K07.6]; other specified disorders of teeth and supporting structures [K08.8]; other specified diseases of jaws [K10.8]; other and unspecified lesions of oral mucosa [K13.7]; glossodynia [K14.6]; pain in joint [M25.5]; pain in throat and chest [R07]; pain in throat [R07.0]; chest pain on breathing [R07.1]; precordial pain [R07.2]; other chest pain [R07.3]; chest pain, unspecified [R07.4]; acute abdomen pain [R10.0]; other and unspecified disturbances of skin sensation [R20.8]; acute pain [R52.0]; other complications of cardiac and vascular prosthetic devices, implants and grafts [T82.8]; other complications of genitourinary prosthetic devices, implants and grafts [T83.8]; other complications of internal orthopaedic prosthetic devices, implants and grafts [T84.8]; priapism [N48.3]; inflammatory conditions of jaws [K10.1]; glossitis [K14.0]; loss of teeth due to accident, extraction or local periodontal disease [K08.1]; burns and corrosions [T20-T32]; frostbite [T33-T35]; wherein the information in brackets refers to the classification according to ICD-10.

In a preferred embodiment, the pain is not classified chronic, but is acute, particularly preferably the pain is acute inflammatory pain.

Preferred causes of nociceptive pain according to the invention include broken or fractured bones, bruises, burns, cuts, inflammation (from infection or arthritis), and sprains. Thus, nociceptive pain includes post-operative pain, cancer pain, low back pain, and inflammatory pain.

In further preferred embodiments of the invention, the pain is selected from the group consisting of pain due to sunburn, prevention of pain prior to tattooing, psoriasis plaques, achilles tendon pain, “Fersensporn”/calcaneussporn, Morton's neuroma, pain/inflammation following tattooing, pain/inflammation following laser treatment of the skin, e.g. for removal of naevus flammeus, plantar fibromatosis, plantar fasciitis; psoriasis, localized; and persistent pain in the skin after radiation.

Typical joint pain symptoms include pain, stiffness, swelling, warmth at joint, weakness, and fatigue.

Typical musculoskeletal pain conditions include back pain, neck pain, tendonitis, myalgia and any pain affecting muscles, tendons, ligaments and bones.

Typical gout pain symptoms include intense joint pain, lingering discomfort, swelling, and redness.

Preferably, the pharmaceutical patch according to the invention is designed for application to the skin for a period of less than 1 day. Thus, according to this embodiment, simultaneous and continuous administration of the Lidocaine constituent and/or Diclofenac constituent can be maintained by removing the used pharmaceutical patch(es) after the predetermined application period has expired and replacing it/them by (a) fresh pharmaceutical patch(es).

As indicated herein before, it is also possible that several pharmaceutical patches according to the invention, e.g. two or three pharmaceutical patches, are simultaneously applied to the skin and simultaneously removed after the application period has expired. For the sake of consiseness, however, this distinction between a single pharmaceutical patch and a plurality of pharmaceutical patches is not made throughout the description. A skilled person recognizes that this embodiment is also within the scope of the invention.

According to a preferred embodiment, the pharmaceutical patch according to the invention is used for a chronic indication, i.e. is for chronic use.

The pharmaceutical patch is designed for application to the skin for an application period, followed by an interruption period (period of discontinued application) during which no pharmaceutical patch is applied to the skin. Thus, intermittent administration of the Lidocaine constituent and/or Diclofenac constituent can be achieved by removing a used pharmaceutical patch after the application period has expired and replacing it by a fresh pharmaceutical patch after the interruption period has expired as well.

The pharmaceutical patch is applied and remains applied to an area of the skin of a patient for an application period of more than about 12 hours, preferably at least about 13 hours, more preferably at least about 14 hours, yet more preferably at least about 15 hours, even more preferably at least about 16 hours, most preferably at least about 17 hours and in particular about 18 hours, but preferably in each case less than about 24 hours (preferably less than about 24 hours per day).

Preferably, after expiry of the application period, the pharmaceutical patch is removed from the skin, and no other pharmaceutical patch is applied to the area of the skin, preferably no other pharmaceutical patch is applied at all, for an interruption period of at least about 1 hour, more preferably at least about 2 hours, still more preferably at least about 3 hours, yet more preferably at least about 4 hours, most preferably at least about 5 hours, and in particular about 6 hours.

Preferably, the application period and the interruption period together last for about 24 hours.

Preferably, the pharmaceutical patch is applied and remains applied to an area of the skin of a patient for an application period of about 18 hours, and after expiry of the application period, the pharmaceutical patch is removed from the skin, and no other pharmaceutical patch is applied to the area of the skin, preferably no other pharmaceutical patch is applied at all, for an interruption period of about 6 hours. It has been found that this regimen is optimized with respect to analgesic effect, patient compliance and tolerability to the skin, especially in long-term treatment.

The locations of the skin, typically human skin, to which the pharmaceutical patch according to the invention is to be applied, are not particularly limited. As the pharmaceutical patch is used for the treatment or prevention of local pain, the pharmaceutical patch is typically applied to a location of the patient's skin that is in close proximity to the origin of the pain to be treated or prevented.

Preferably, the pharmaceutical patch according to the invention is applied to the skin of the breast, or the skin of the knee, or the skin of the elbow, or the skin of the hip, or the skin of the hand, or the skin of the spine, or the skin of the back, particularly of the lower back.

In a preferred embodiment, the pharmaceutical patches according to the invention are repeatedly applied to the same location on the skin, i.e. after a first pharmaceutical patch has been used and needs to be replaced by a second pharmaceutical patch in order to maintain the desired pharmacological effect, said second pharmaceutical patch is preferably applied to the same location on the skin to which said first pharmaceutical patch was applied before.

The intended duration of application is preferably at least 1, 2, 3, 4, 5, 6, or 7 days but can be longer, principally unlimited, ranging from provisional to prolonged use in acute or chronic conditions. In preferred embodiments, the intended duration of application is preferably at least 8, 9, 10, 11, 12, 13, or 14 days. In preferred embodiments, the intended duration of application is preferably at least 2, 3, 4, 5, 6, 7, 8, 9, 10, 11, 12, 13, 14, 15, or 16 weeks. Preferably, on every day of such regimen, a pharmaceutical patch is applied to the human skin for about 18 hours (application period) followed by a break of about 6 hours (interruption period), before a new patch is applied, preferably to the same area of the skin.

The pharmaceutical patch according to the invention is for administration to the skin of a mammal, preferably of a human (pediatrics or adults).

In a particularly preferred embodiment of the invention, the intended recipient of the treatment is a patient with renal and/or hepatic impairment.

Subjects suffering from moderate to severe pain may have impaired hepatic and/or renal function for various reasons such as genetic disposition, acquired liver and/or kidney disease, or side effect of a medication that is administered for treating another primary disorder or disease or the same disorder or disease. For example, it is known that NSAIDs may cause renal impairment.

Liver function tests are routinely performed and give information about the state of a subject's liver. Results of hepatic tests may be associated with cellular integrity, functionality, and conditions linked to the biliary tract. These tests can be used to detect the presence of liver disease, distinguish among different types of liver disorders, gauge the extent of known liver damage, and follow the response to treatment. Hepatic insufficiency can be quantified using any of a number of scales including a model end stage liver disease (MELD) score, a Child-Pugh score, or a Conn score. The Child-Pugh score employs two clinical features (encephalopathy and ascites) and three laboratory-based parameters (S-albumin, 5-bilirubin and prothrombin time). Each measure is scored with 1 to 3 points, with 3 points indicating most severe derangement. The points for all five items are added and liver function is then classified into Child-Pugh classes A to C.

Similarly, kidney function tests are routinely performed and give information about the state of a subject's kidneys. Renal failure is a medical condition in which the kidneys fail to adequately filter waste products from the blood. Renal failure is mainly determined by a decrease in glomerular filtration rate (GFR), the rate at which blood is filtered in the glomeruli of the kidney. This is detected by a decrease in or absence of urine production or determination of waste products (e.g. creatinine) in the blood. GFR can be calculated from creatinine concentration in blood, creatinine concentration in urine, and volume of urine collected over 24 hours. However, in clinical practice, estimates of creatinine clearance based on the serum creatinine level are routinely used to measure GFR (eGFR) according to various formulas.

Many analgesics must not be administered to or are not recommended for subjects with impaired hepatic or renal function or at least require specific attention and care during treatment.

The liver plays a central role in the pharmacokinetics of the majority of drugs. Liver dysfunction may not only reduce the blood/plasma clearance of drugs eliminated by hepatic metabolism or biliary excretion, it can also affect plasma protein binding, which in turn could influence the processes of distribution and elimination. Portal-systemic shunting, which is common in advanced liver cirrhosis, may substantially decrease the presystemic elimination (i.e., first-pass effect) of high extraction drugs following their oral administration, thus leading to a significant increase in the extent of absorption. Chronic liver diseases are associated with variable and non-uniform reductions in drug-metabolizing activities. Subjects with liver cirrhosis are more sensitive to the central adverse effects of opioid analgesics (R. K. Verbeeck, Eur J Clin Pharmacol. 2008, 64(12), 1147-61).

In subjects with renal impairment dose adjustment is often necessary for drugs eliminated by renal excretion. The treatment of pain in subjects with impaired renal or hepatic function may also be problematic. In the presence of renal failure, significant changes occur in the metabolism and pharmacokinetics of these drugs, which can lead to adverse reactions due to the accumulation of parental compounds and active or toxic metabolites (P. Niscola et al., G Ital Nefrol. 2011, 28(3), 269-77).

In consequence, pain therapy in subjects with hepatic or renal impairment is often difficult and conventional analgesia is not always applicable. Thus, there is a demand for analgesics that are well tolerated, have no or only few side effects, and may be administered to subjects with impaired hepatic or renal function even without the need for dosing adjustments.

Hepatic and renal function can be easily assessed by a skilled person and are subject to routine analysis. A skilled person can easily and clearly distinguish a subject having impaired hepatic function and/or impaired renal function from a subject having no impaired hepatic function and no impaired renal function, respectively.

The degree of the impairment of the hepatic function of the subject may be mild, moderate or severe. Preferably, the impairment of the hepatic function is at least mild, or at least moderate, or severe. In this regard, “at least mild” encompasses mild, moderate and severe, whereas “at least moderate” encompasses moderate and severe.

In a preferred embodiment, the impairment of the hepatic function is according to the Child-Pugh Score such that depending upon the degree of hepatic impairment the subjects may be classified in any one of classes A (mild), B (moderate) or C (severe) according to the Child-Pugh Score:

| Assessment | Degree of abnormality | Score |
|---|---|---|
| Encephalopathy | None | 1 |
| | Moderate | 2 |
| | Severe | 3 |
| Ascites | Absent | 1 |
| | Slight | 2 |
| | Moderate | 3 |
| Bilirubin | <2 | 1 |
| [mg/dL] | 2.1 to 3 | 2 |
| | >3 | 3 |
| Albumin | >3.5 | 1 |
| [g/dL] | 2.8 to 3.5 | 2 |
| | <2.8 | 3 |
| Prothrombin Time | 0 to 3.9 | 1 |
| [seconds > control] | 4 to 6 | 2 |
| | >6 | 3 |

| Total Score | Class | Severity |
|---|---|---|
| 5 to 6 | A | Mild |
| 7 to 9 | B | Moderate |
| 10 to 15 | C | Severe |

This categorization according to the Child-Pugh classification is in line with the respective EMA Guideline (Guideline on the evaluation of the pharmacokinetics of medicinal products in subjects with impaired hepatic function, 17 Feb. 2005, CPMP/EWP/2339/02) and FDA Guidance (Guidance for Industry—Pharmacokinetics in subjects with impaired hepatic function: Study design, data analysis, and impact on dosing and labeling, May 2003).

Preferably, the impairment of the hepatic function of the subject is of class A, B or C according to the Child-Pugh Score.

In preferred embodiments, the subject is classified by a total score according to the Child-Pugh classification of at least 5, or at least 6, or at least 7, or at least 8, or at least 9, or at least 10, or at least 11, or at least 12, or at least 13, or at least 14, or 15.

The causes of the impaired hepatic function are not particularly limited and include genetic disposition (e.g. inborn metabolic disorders and the like), acquired liver disease (e.g. diseases due to infection such as hepatitis, due to toxic substances such as alcohol, steatohepatosis, and the like), or side effect of a medication that is administered for treating another primary disorder or disease (e.g. chemotherapy, NSAIDs, and the like).

The degree of the impairment of the renal function of the subject may be mild, moderate or severe. Preferably, the impairment of the renal function, preferably in terms of decrease in estimated glomerular filtration rate (eGFR), is at least mild, or at least moderate, or severe. In this regard, “at least mild” encompasses mild, moderate and severe, whereas “at least moderate” encompasses moderate and severe.

As mentioned above, renal impairment may be described qualitatively and quantitatively by various classification systems. Preferably, no dose adaptation is required when applying the pharmaceutical patch according to the invention to subjects across the full range of renal impairment according to the invention such that the classification system used is not relevant.

In a preferred embodiment, the impairment of the renal function is based on the estimated creatinine clearance (Cl_{CR}) by the Cockcroft-Gault equation or on the estimated glomerular filtration rate (eGFR) from the Modification of Diet in Renal Disease (MDRD). Cockcrof-Gault and eGFR are two commonly used serum-creatinine based equations. Depending upon the degree of renal impairment the subjects may be classified in any one of stage 1 (normal), stage 2 (mild), stage 3 (moderate), stage 4 (severe) or stage 5 (end stage renal disease) according to the following classification of renal function based on eGFR or CL_{Cr}:

| | | eGFR [mL/ | CLCr |
|---|---|---|---|
| Stage | Description | min/1.73 m2) | [mL/min] |
| 1 | Normal GFR | ≥90 | ≥90 |
| 2 | Mild decrease in GFR | 60 to 89 | 60 to 89 |
| 3 | Moderate decrease in GFR | 30 to 59 | 30 to 59 |
| 4 | Severe decrease in GFR | 15 to 29 | 15 to 29 |
| 5 | End Stage Renal Disease (ESRD) | <15 not on | <15 not on |
| | | dialysis | dialysis |
| | | requiring | requiring |
| | | dialysis | dialysis |

This categorization according to eGFR or CL_{Cr} is in line with the respective FDA Guidance (Guidance for Industry—Pharmacokinetics in subjects with impaired renal function: Study design, data analysis, and impact on dosing and labeling, draft guidance, March 2010, Revision 1). According to the invention different threshold values for mild, moderate and severe impairment of renal function may apply to specific subgroups of subjects, e.g. in pediatric subjects. These different threshold values are known to the skilled person and preferably are in accordance with the current FDA Guidance.

Preferably, the impairment of the renal function of the subject is of stage 2, 3 or 4 according to the estimated glomerular filtration rate eGFR or the creatinine clearance Cl_{Cr}.

In preferred embodiments, the subject is classified by an eGFR and a CL_{Cr}, respectively, of less than 90, or not more than 85, or not more than 80, or not more than 75, or not more than 70, or not more than 65, or not more than 60, or not more than 55, or not more than 50, or not more than 45, or not more than 40, or not more than 35, or not more than 30, or not more than 25, or not more than 20, or not more than 15, in either case mL/min/1.73 m² and mL/min, respectively.

The causes of the impaired renal function are not particularly limited and include genetic disposition, acquired kidney disease (e.g. chronic kidney disease, due to diabetes, arterial hypertension, infection), or side effect of a medication that is administered for treating another primary disorder or disease (e.g. chemotherapy, NSAIDs).

Another aspect of the invention relates to a kit comprising a plurality of pharmaceutical patches as defined above for use in the local treatment or prevention of pain,
wherein a first pharmaceutical patch is applied and remains applied to an area of the skin of a patient for a first application period of more than about 12 hours, more preferably at least about 13 hours, still more preferably at least about 14 hours, yet more preferably at least about 15 hours, even more preferably at least about 16 hours, most preferably at least about 17 hours and in particular about 18 hours, but preferably in each case less than about 24 hours;
wherein after expiry of the first application period, the first pharmaceutical patch is removed from the skin, and no other pharmaceutical patch is applied to the same area of the skin, preferably no other pharmaceutical patch is applied at all, for a first interruption period of at least about 1 hour, more preferably at least about 2 hours, still more preferably at least about 3 hours, yet more preferably at least about 4 hours, most preferably at least about 5 hours, and in particular about 6 hours;
wherein after expiry of the first interruption period, a second pharmaceutical patch is applied and remains applied to the same area of the skin of the patient for a second application period of more than about 12 hours, more preferably at least about 13 hours, still more preferably at least about 14 hours, yet more preferably at least about 15 hours, even more preferably at least about 16 hours, most preferably at least about 17 hours and in particular about 18 hours, but preferably in each case less than about 24 hours; and wherein after expiry of the second application period, the second pharmaceutical patch is removed from the skin, and no other pharmaceutical patch is applied to the same area of the skin, preferably no other pharmaceutical patch is applied at all, for a second interruption period of at least about 1 hour, more preferably at least about 2 hours, still more preferably at least about 3 hours, yet more preferably at least about 4 hours, most preferably at least about 5 hours, and in particular about 6 hours.

Preferably, the first application period and the second application period are each about 18 hours, and wherein the first interruption period and the second interruption period are each about 6 hours.

Preferably, the kit according to the invention comprises at least 1, 2, 3, 4, 5, 6, or 7 pharmaceutical patches according to the invention. In preferred embodiments, the kit according to the invention comprises at least 8, 9, 10, 11, 12, 13, or 14 pharmaceutical patches according to the invention. In preferred embodiments, the kit according to the invention comprises at least 14, 21, 28, 35, 42, 49 or 56 pharmaceutical patches according to the invention. Preferably, each and every pharmaceutical patch according to the invention that is contained in the kit according to the invention is to be applied to the human skin for about 18 hours (application period) followed by a break of about 6 hours (interruption period), before a new patch is applied, preferably to the same area of the skin.

The following examples further illustrate the invention but are not to be construed as limiting its scope.

### EXAMPLES

An interaction study was performed as a single dose study in the CFA-hindpaw inflammation, paw pressure test.

### Part A—Intraperitoneal Injection

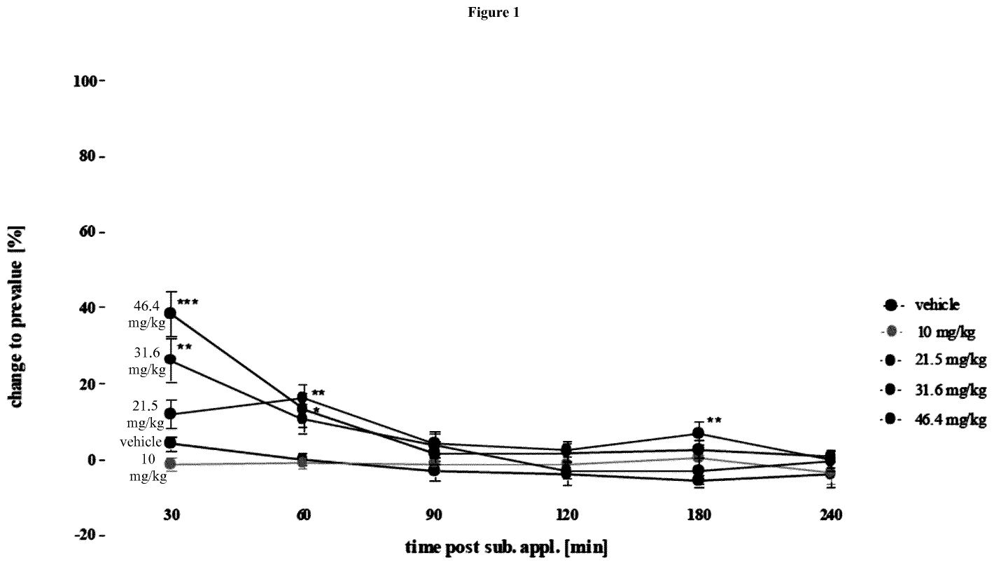

In comparative example 1 and inventive examples 1 to 4 described below, a compound or combination of compounds was administered by intraperitoneal injection (FIGS. 1 to 9).

### Comparative Example 1—Separate Administration

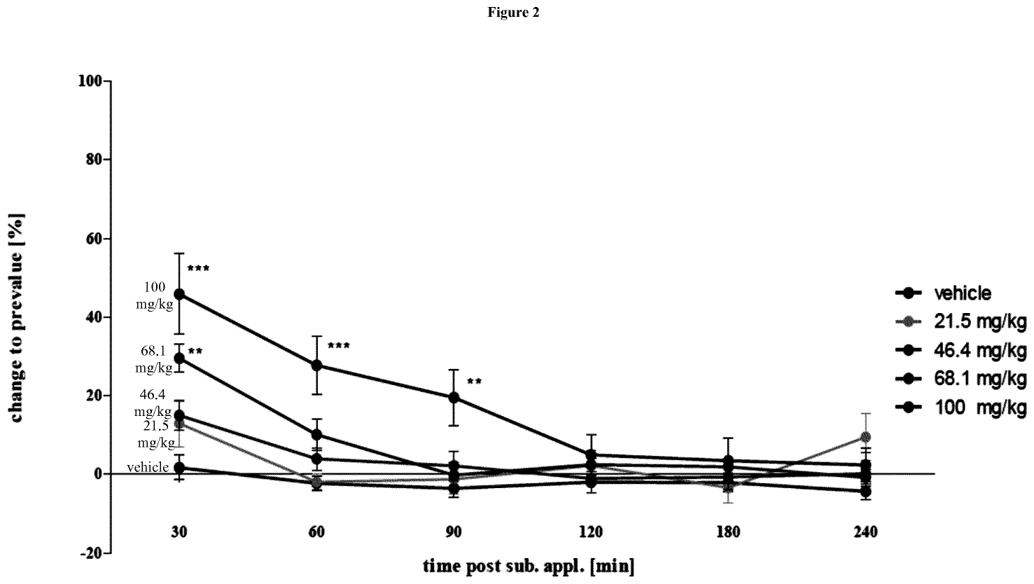

A dose dependent efficacy of both compounds was observed when they were administered alone. Lidocaine showed dose dependent efficacy at a dose range of from 10 mg/kg to 46.4 mg/kg; ip (FIG. 1) and Diclofenac showed dose dependent efficacy at a dose range of from 21.5 mg/kg to 100 mg/kg; ip (FIG. 2).

### Inventive Example 1—Combined Administration (Ratio Lidocaine:Diclofenac=1:3.17)

A combination of Lidocaine:Diclofenac (21.5 mg/kg:68.1 mg/kg) was tested:
Lidocaine/Diclofenac in CFA-PPT (% change to prevalue)
Dose ratio (Lidocaine/Diclofenac): 21.5 mg/kg:68.1 mg/kg=1:3.17

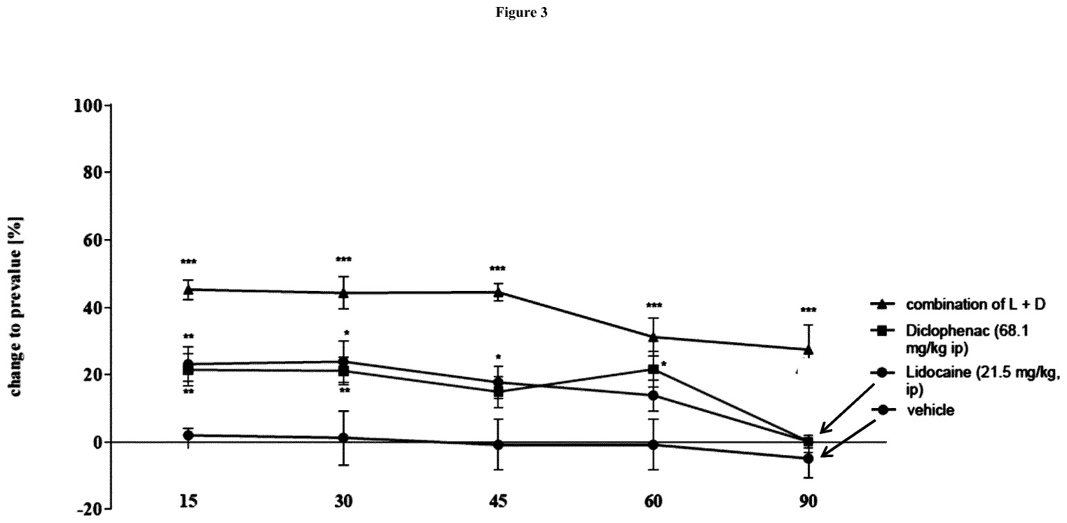

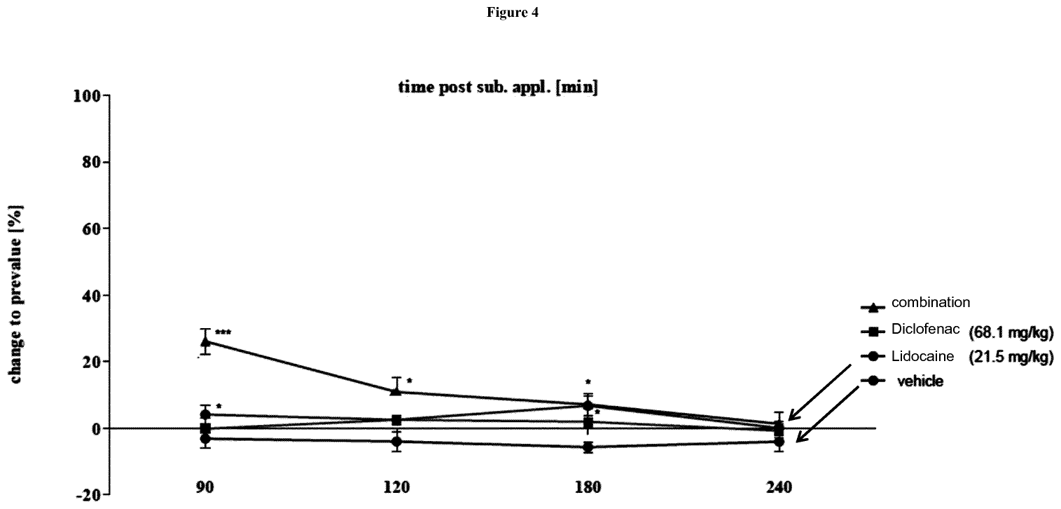

The results for the time window of 0 to 90 min after administration are shown in FIG. 3. The results for the time window of 90 to 240 min after administration are shown in FIG. 4.

The experiment revealed that, under the given conditions, the intraperitoneal administration of Diclofenac (68.1 mg/kg; ip) and Lidocaine (21.5 mg/kg, ip) showed similar efficacy in the range of 20-25% when given alone. The combination of Diclofenac and Lidocaine showed additive interaction from 15 to 60 min post administration and showed a supra-additive (i.e. synergistic) effect at 90 min. This study showed again synergistic action 90 min after administration and probably 120 min after administration.

### Inventive Example 2—Combined Administration (Dose Ratio Lidocaine:Diclofenac=1:2.16)

A combination of Lidocaine:Diclofenac (31.6 mg/kg:68.1 mg/kg) was tested:
Lidocaine/Diclofenac in CFA-PPT (% change to prevalue)
Dose ratio (Lidocaine/Diclofenac): 31.6 mg/kg:68.1 mg/kg=1:2.16

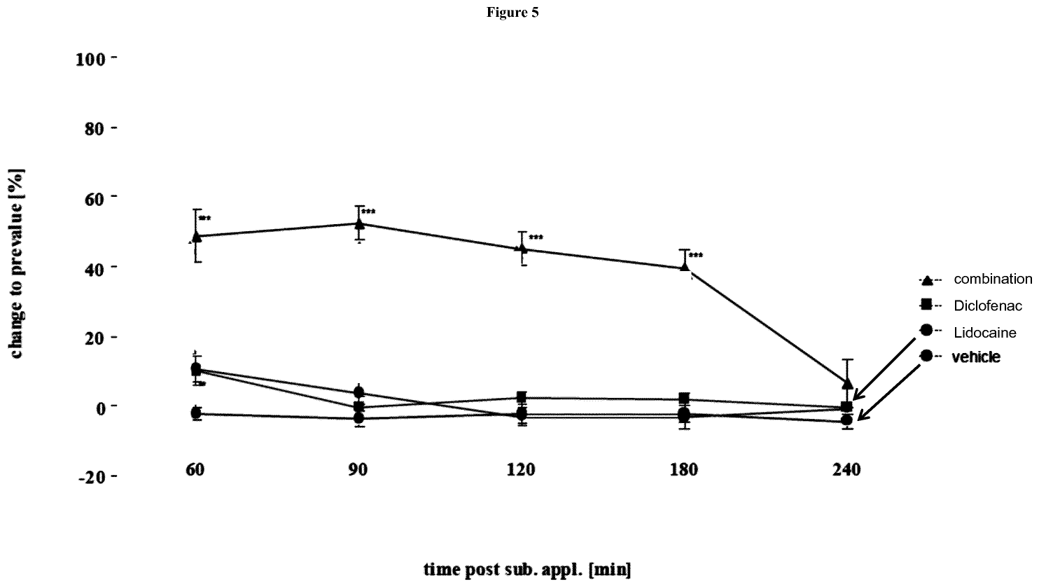

The results are shown in FIG. 5. The combination of Lidocaine:Diclofenac in a ratio of 31.6 mg/kg:68.1 mg/kg=1:2.16 showed synergistic action 60 min, 90 min, 120 min and 180 min after administration.

### Inventive Example 3—Combined Administration (Dose Ratio Lidocaine:Diclofenac=2.16:1)

A combination of Lidocaine:Diclofenac (46.4 mg/kg:21.5 mg/kg) was tested:
Lidocaine/Diclofenac in CFA-PPT (% change to prevalue)
Dose ratio (Lidocaine/Diclofenac): 46.4 mg/kg:21.5 mg/kg=2.16:1

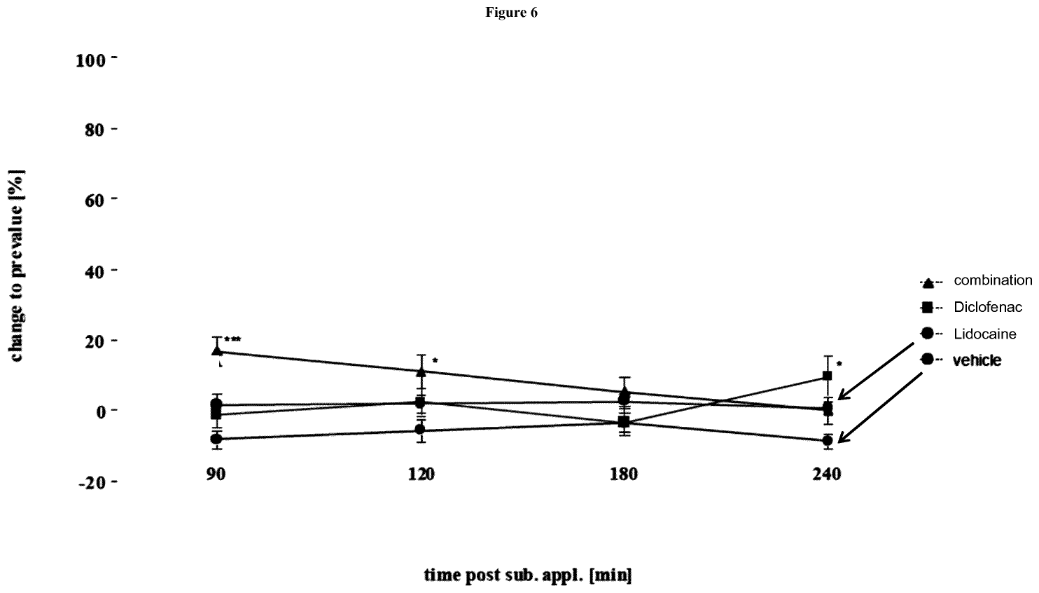

The results are shown in FIG. 6. Lidocaine:Diclofenac in a dose ratio of 46.4 mg/kg:21.5 mg/kg=2.16:1 showed synergistic action 90 min and 120 min after administration.

### Inventive Example 4—combined administration (ratio Lidocaine:Diclofenac=1:1.47)

A combination of Lidocaine:Diclofenac (31.6 mg/kg:46.4 mg/kg) was tested:
Lidocaine/Diclofenac in CFA-PPT (% change to prevalue)
Dose ratio (Lidocane/Diclofenac): 31.6 mg/kg:46.4 mg/kg=1:1.47

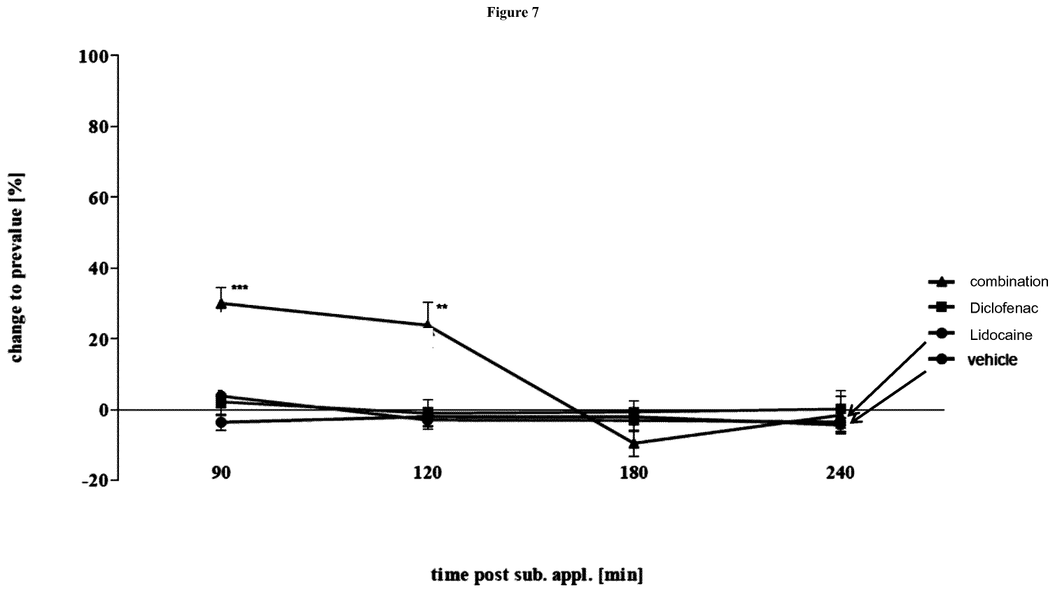

The results are shown in FIG. 7. Lidocaine:Diclofenac in a dose ratio of 31.6 mg/kg:46.4 mg/kg=1:1.47 showed synergistic action 90 min and 120 min after administration.

### Summary of the Experiments with Intraperitoneal Administration

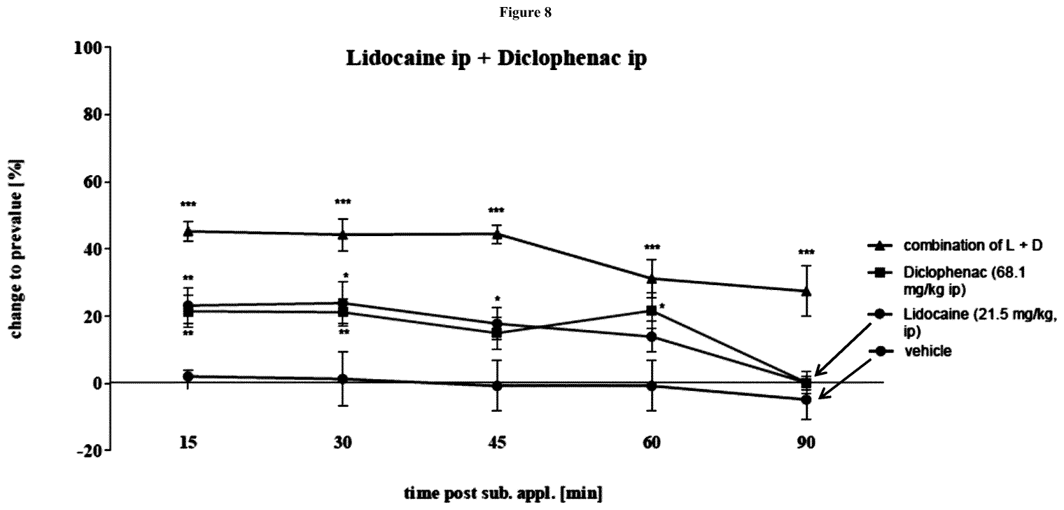

Intraperitoneal administration of Lidocaine (21.5 mg/kg, ip) and Diclofenac (68.1 mg/kg; ip) showed similar efficacy in the range of 20-25% when given alone. The combination of Lidocaine and Diclofenac showed additive interaction from 15 to 60 min post administration and showed a supra-additive (i.e. synergistic) effect at 90 min. As shown in FIG. 8, intraperitoneal administration of Lidocaine (21.5 mg/kg, ip) and Diclofenac (68.1 mg/kg; ip) showed similar efficacy in the range of 20-25% when given alone.

The combination of Diclofenac and Lidocaine showed additive interaction from 15 to 60 min post administration and showed a supra-additive effect at 90 min.

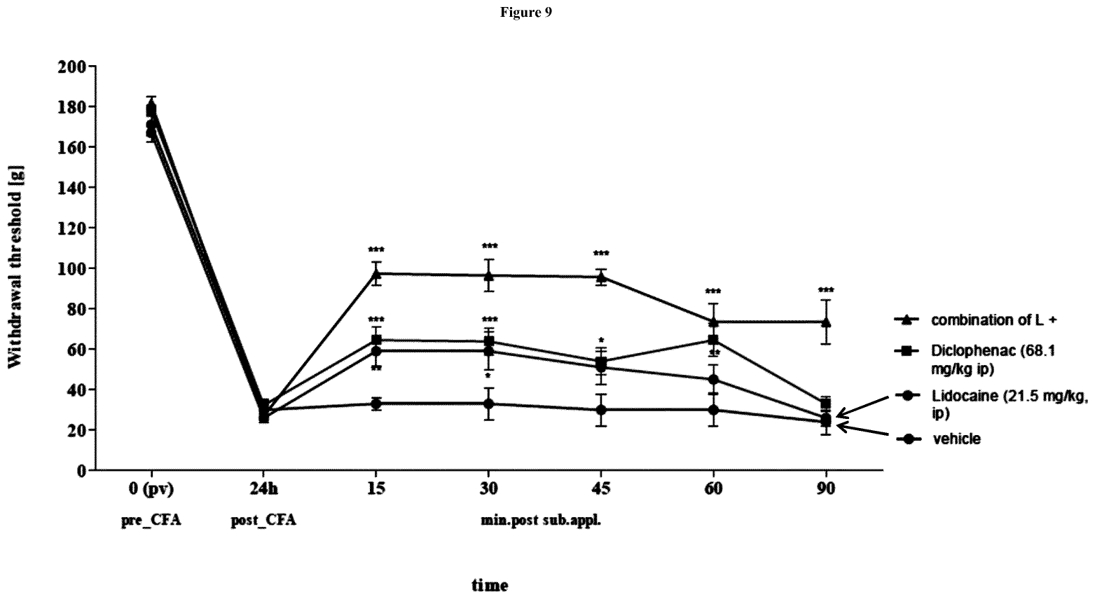

Lidocaine/Diclofenac in CFA-PPT (withdrawal thresholds) are shown in FIG. 9.

### Part B—Topical Application to the Skin

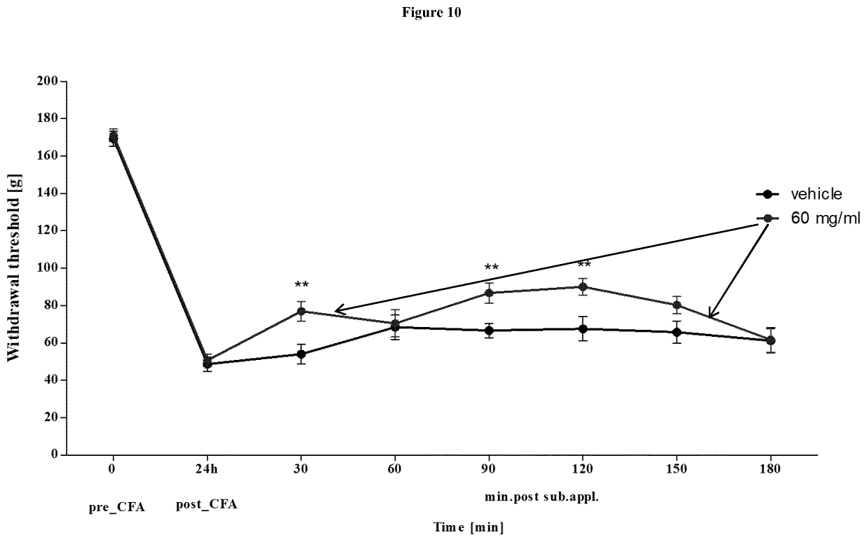

In comparative example 2 and inventive examples 5 to 8 as described below, a compound or combination of compounds was administered topically to the paw by means of an ointment or solution (FIGS. 10 to 18).

The efficacy was tested when a compound or a compositions of compounds was administered topically as an ointment or a solution. The study was performed on male albino rats (150-180 g body weight) to which an edema of paw was induced by injection of Complete Freunds Adjuvant (CFA). CFA is a composition of inactivated and dried Myobacteria tuberculoses, emulsified in mineral oil which is used as an immunopotentiator causing a painful reaction that lasts 7-8 days after subcutaneous injection.

The studies of comparative example 2 and inventive examples 5 to 8 were performed according to the following procedure: 50 μL of CFA were injected sub plantar into the right hind paw, inducing mechanical-hyperalgesia after 24 hours. During a short anaesthesia an ointment comprising one compound or a combination of compounds was administered to the right hind paw and the paw was wrapped with polyethylene (PE)-foil and a smooth tape to prevent licking of the paw. The formulation of the vehicle was 4% hydroxypropylmethylcellulose (HPMC)/10% dimethylsulfoxide (DMSO) and aqua ini.

The pain was measured by pressing a pointer on the inflamed paw. The pointer exerts a force, increasing at a constant rate, monitored by a linear scale. Pain is deemed to be perceived one the rat starts to withdraw the paw. This value (withdrawal threshold in gram) is the pressure at which the animal feels pain (cut-off 450 g). Efficacy of the test compounds is expressed as a percent change of withdrawal threshold after compound administration to the pre-value (withdrawal threshold before compound administration).

### Comparative Example 2—Separate Administration

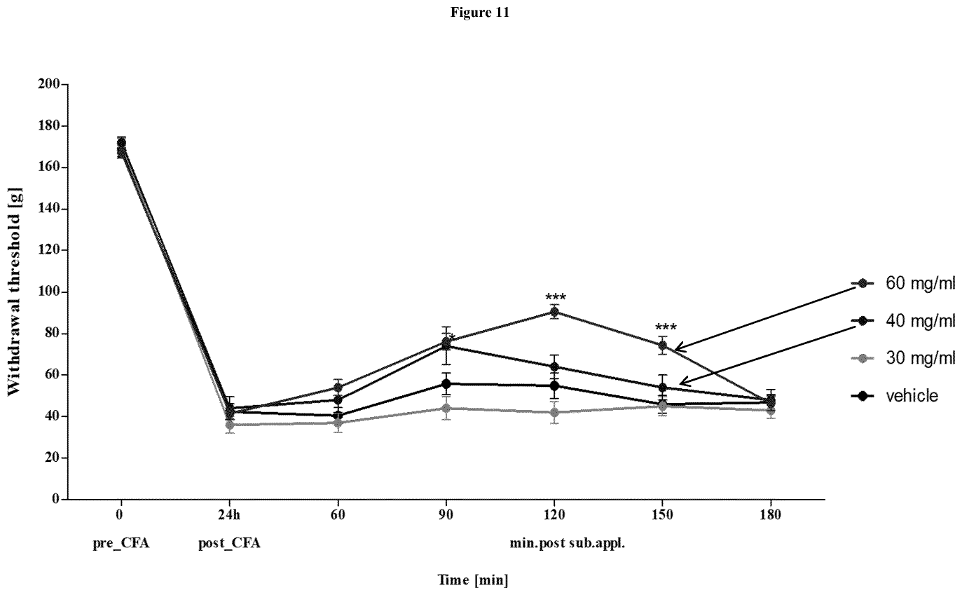

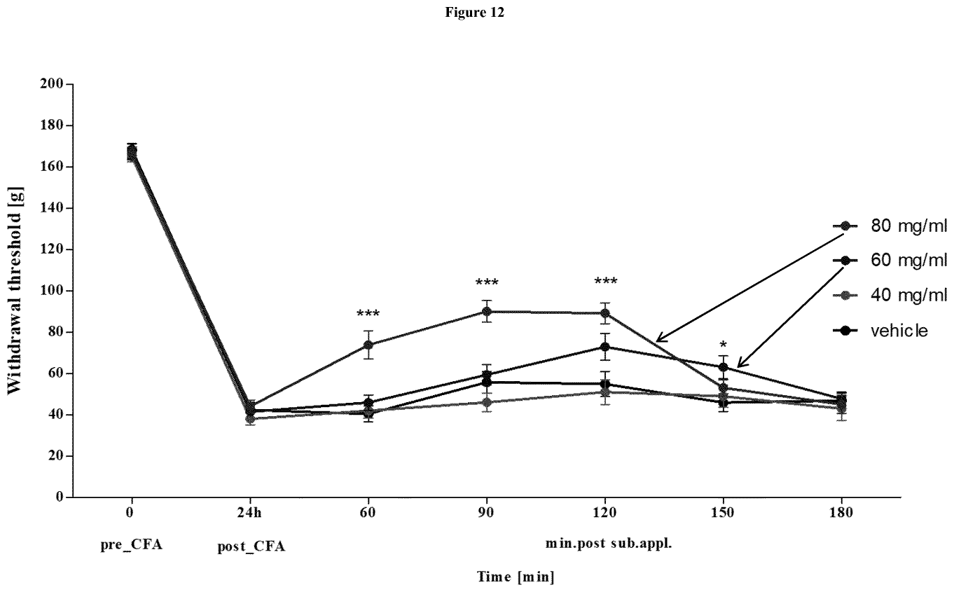

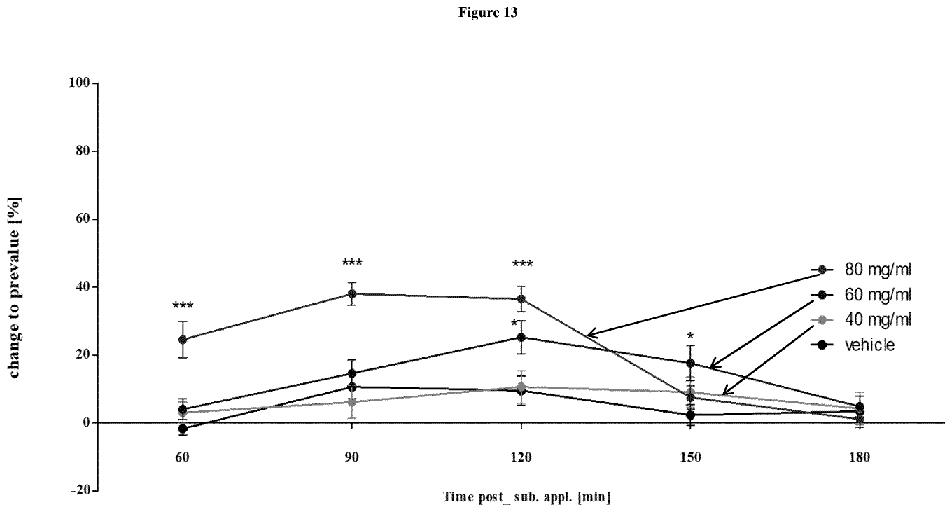

One hour was identified as an optimal incubation time (shown exemplary data for Lidocaine in FIG. 10). A dose dependent efficacy of both individual compounds was observed when topically administered. Lidocaine showed dose dependent efficacy at a dose range from 30 mg/ml to 60 mg/ml (FIG. 11) and Diclofenac showed dose dependent efficacy at a dose range from 40 mg/ml to 80 mg/ml (FIGS. 12 and 13).

### Inventive Example 5—Combined Administration (Concentration Ratio Diclofenac:Lidocaine=1.5:1)

A combination of Diclofenac:Lidocaine=1.5:1 (60 mg/ml: 40 mg/ml) was tested:
Diclofenac/Lidocaine in CFA-PPT (% change to pre-value)
Concentration ratio (Diclofenac/Lidocaine): 60 mg/ml: 40 mg/ml=1.5:1

The experiment revealed that under the given conditions topical administration of Diclofenac (60 mg/ml) and Lidocaine (40 mg/ml) after 90 min. showed similar efficacy in the range of 15-25% when given alone. The combination of Diclofenac and Lidocaine showed additive interaction from 90 to 120 min. post administration and showed a supra-additive (i.e. synergistic) effect from 120 to 180 min.

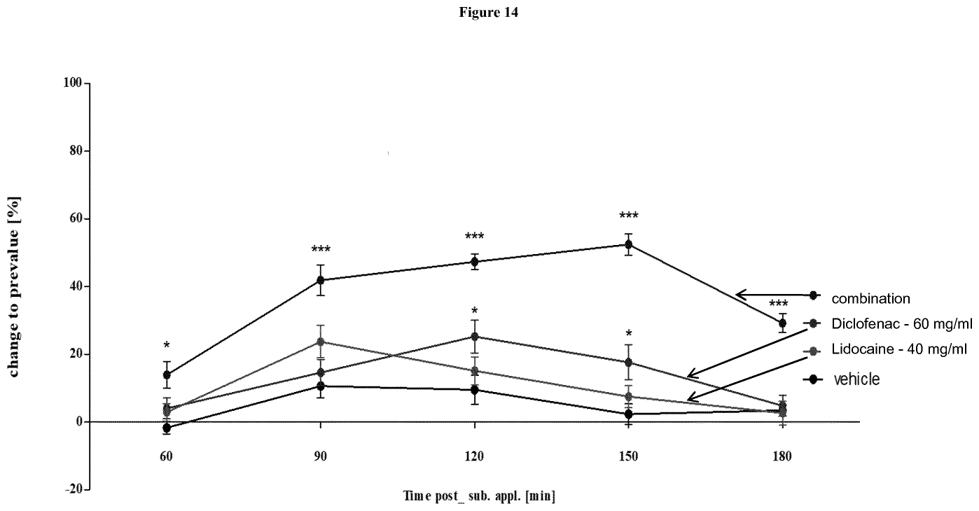

The results for the time window of 60 to 180 min after administration are shown in FIG. 14.

### Inventive Example 6—Combined Administration (Concentration Ratio of Diclofenac:Lidocaine=2:1)

Two combinations of Diclofenac:Lidocaine=concentration ratio of 2:1 (60 mg/ml: 30 mg/ml and 80 mg/ml: 40 mg/ml) were tested:
Diclofenac/Lidocaine in CFA-PPT (% change to prevalue)
Concentration ratio (Diclofenac/Lidocaine): 60 mg/ml: 30 mg/ml=2:1 and 80 mg/ml: 40 mg/ml=2:1

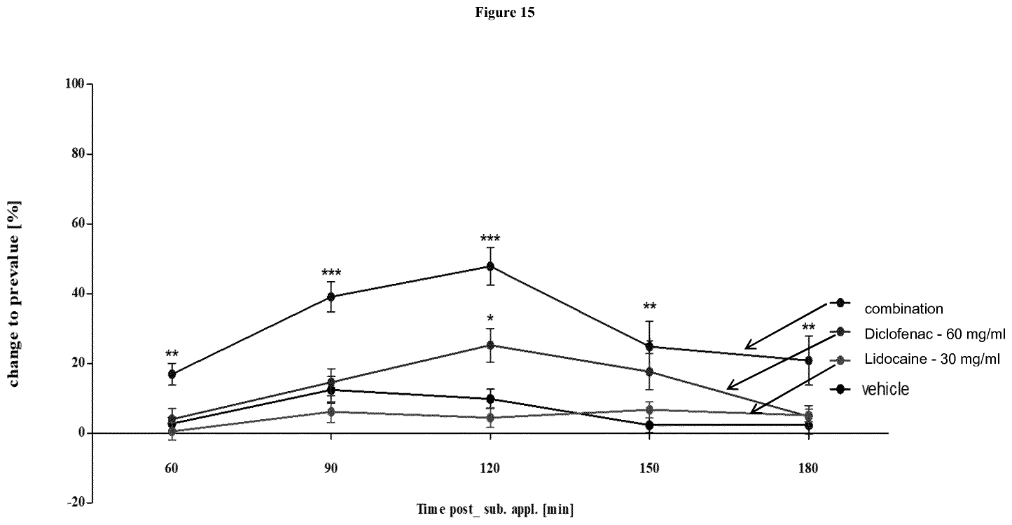

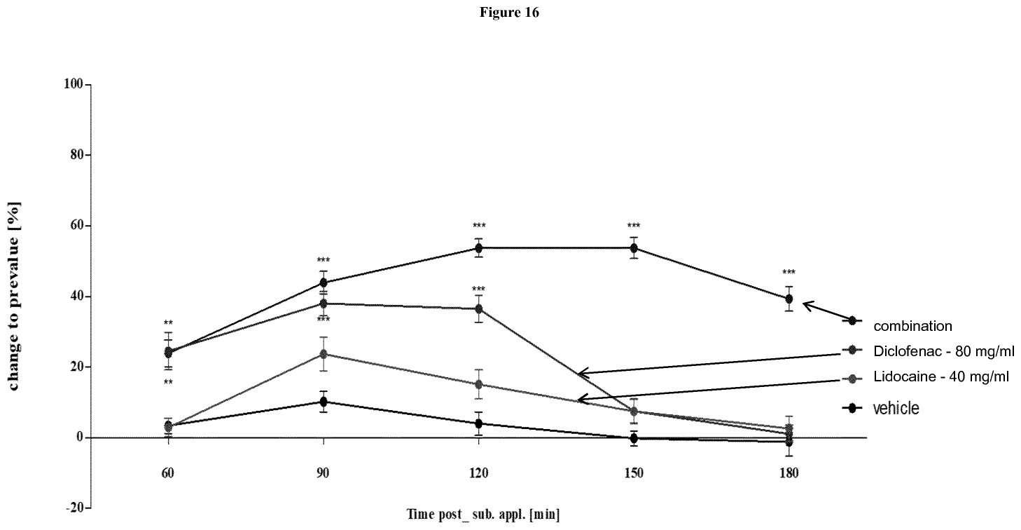

The combination of Diclofenac:Lidocaine in a concentration ratio of 60 mg/ml: 30 mg/ml=2:1 showed synergistic action 60 min, 90 min, 120 min and 180 min after administration (FIG. 15). The combination of Diclofenac:Lidocaine in a ratio of 80 mg/ml: 40 mg/ml=2:1 showed synergistic action 150 min and 180 min after administration (FIG. 16).

The efficacy of the combination of Diclofenac:Lidocaine in a concentration ratio of 80 mg/ml: 40 mg/ml=2:1 at 150 min and 180 min was substantially higher with values from around 40% to 50%, compared to the administration of 80 mg/ml of Diclofenac alone or 40 mg/ml of Lidocaine alone in the same time window which both showed an efficacy of under 15%.

### Inventive Example 7—Combined Administration (Concentration Ratio Diclofenac:Lidocaine=2.7:1)

A combination of Diclofenac:Lidocaine=concentration ratio of 2.7:1 (80 mg/ml: 30 mg/ml) was tested:
Diclofenac/Lidocaine in CFA-PPT (% change to prevalue)
Concentration ratio (Diclofenac/Lidocaine): 80 mg/ml: 30 mg/ml=2.7:1

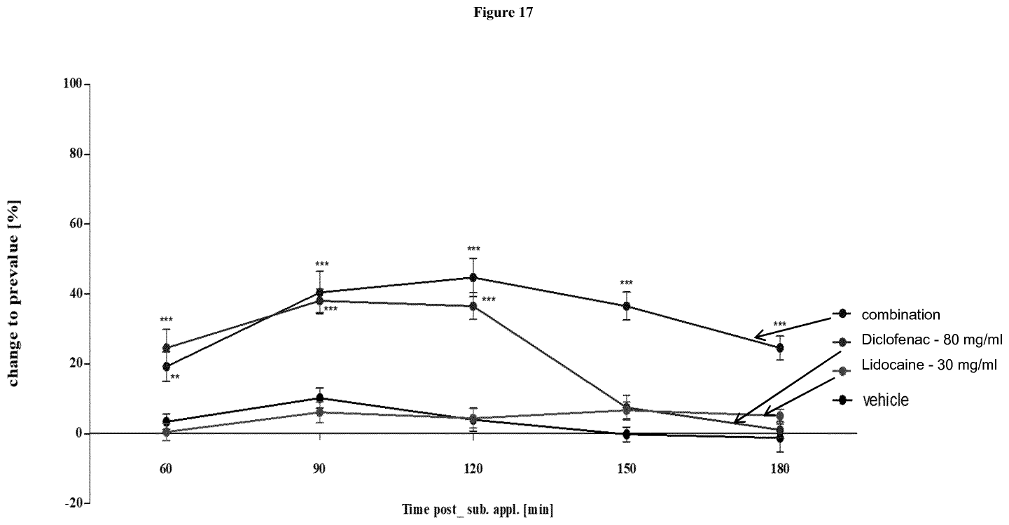

The results are shown in FIG. 17. Diclofenac:Lidocaine in a concentration ratio of 80 mg/ml: 30 mg/ml=2.7:1 showed synergistic action 150 min and 180 min after administration.

### Inventive Example 8—Combined Administration (Concentration Ratio Diclofenac:Lidocaine=1:1)

A combination of Diclofenac:Lidocaine=1:1 (40 mg/ml: 40 mg/ml) was tested:
Diclofenac/Lidocaine in CFA-PPT (% change to prevalue)
Ratio (Diclofenac/Lidocaine): 40 mg/ml: 40 mg/ml=1:1

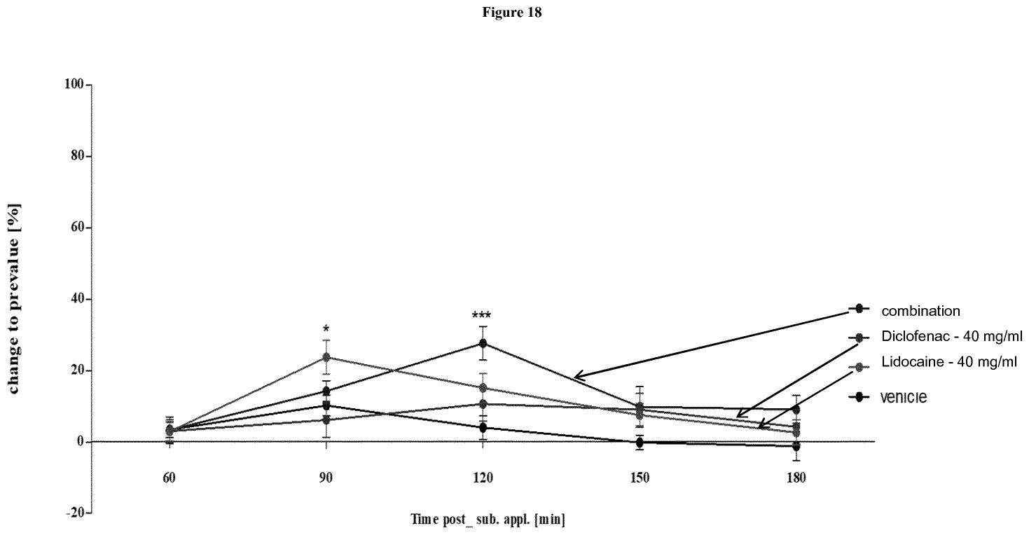

The results are shown in FIG. 18. Diclofenac/Lidocaine: 40 mg/ml: 40 mg/ml=1:1 (concentration ratio) showed better efficacy 120 min after administration.

Summary of the Experiments with Topical Application to the Skin

When applied topically the combinations of Diclofenac/Lidocaine at concentration ratios of 1.5:1, 2.2 and 2.7:1 showed synergistic interaction. A supraadditive effect mostly occurred at later timepoints, i.e. after at least 120 min post administration.

The combination of Diclofenac and Lidocaine according to the invention unexpectedly provides significant therapeutic superiority compared to the single agents due to a pharmacological interaction. Surprisingly, the addition of increasing doses of Lidocaine bestowed both a long-term pharmacological potentiation of Diclofenac and a coalistic pharmacological effect (neither drug active individually) across the diverse experimental testing.

### Inventive Example 8—Pharmaceutical Patch

A typical pharmaceutical patch according to the invention has a contact surface to the skin of 140 cm² and is composed of a surface layer (backing) of non-woven polyethylene terephthalate, an adhesive layer comprising the Diclofenac constituent and the Lidocaine constituent in a hydrogel, and a removable release liner.

Typical ingredients of the adhesive paste forming the adhesive layer are compiled in the table here below:

Lidocaine (700 mg)
Diclofenac epolamine (182 mg)
methyl parahydroxybenzoate
propyl parahydroxybenzoate
propylene glycol
sorbitol
glycerol
polyacrylic acid
sodium polyacrylate
sodium carmellose
heavy kaolin
aluminum glycinate
sodium EDTA
tartaric acid
purified water
urea

### Inventive Example 9—Pharmaceutical Patch

The relative systemic bioavailability of lidocaine and diclofenac of the patch according to inventive example 8 was compared to commercial patches only containing lidocaine (Versatis®) and only containing diclofenac (Flector® Tissugel). The relative systemic bioavailability of lidocaine and diclofenac after a single 18-hour application in the lumbar region of the fixed-dose combination patch containing lidocaine 5% and diclofenac epolamine 1.3% compared to Versatis® and Flector® Tissugel patches was assessed in healthy adult subjects. The clinical trial was designed as a randomized, single-site, open-label, single application, 3-way crossover, exploratory Phase I trial in healthy male and female subjects. The primary objective was to assess the relative systemic bioavailability of lidocaine and diclofenac released from the inventive patch and from the marketed patches Versatis® and Flector® Tissugel (diclofenac patch) following single application. Furthermore data were collected on skin irritation and patch adhesiveness. Bioequivalence assessment was not intended in this trial.

PK sampling was performed until 58 hours after patch application (=40 hours after patch removal). Subject disposition—safety set: 22 subjects, PK set: 22 subjects. Enrollment Visit—Day 1—Allocation to treatment sequences—Treatment Period 1 (4 days), Treatment Period 2 (4 days) or Washout Phase 3-10 days between IMP applications—Treatment Period 3 (4 days)—Final Visit within 7-14 days after Treatment Period 3. In each treatment period, a single patch was applied for 18 hours, hospitalization from Day 1 until 40 hours after patch removal.

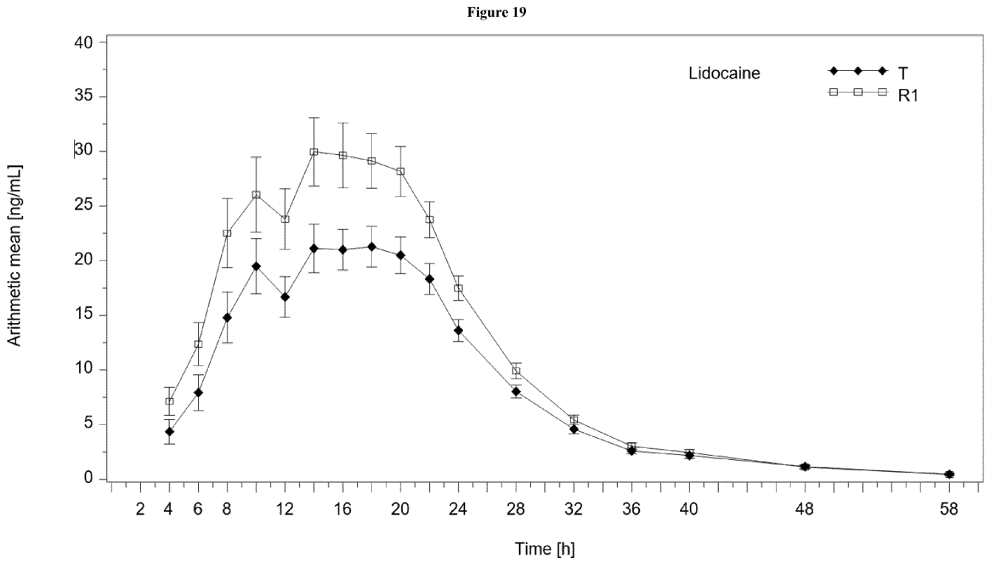

The mean plasma-concentration-time profiles of lidocaine following 18 h application of the patch according to the invention (T) and Versatis® (R1)—PK Set are shown in FIG. 19. It can be concluded that the time course of PK time profile for both patches was similar with an overall low exposure but continuously lower for the patch according to the invention.

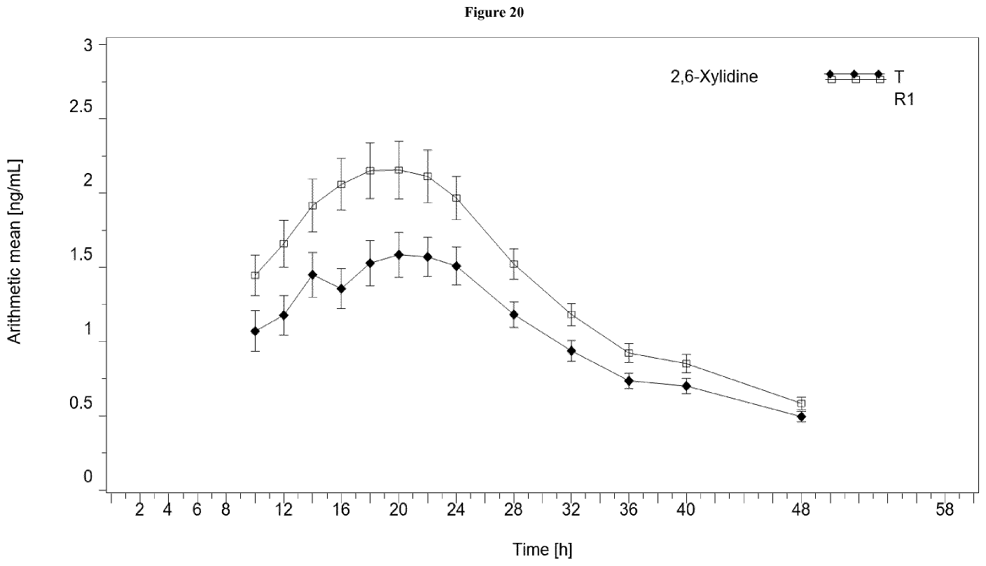

The mean plasma-concentration-time profiles of 2,6-xylidine (metabolite of lidocaine) following 18 h application of the patch according to the invention (T) and Versatis® (R1)—PK Set are shown in FIG. 20. It can be concluded that the time course of PK time profile for both patches was similar with an overall low exposure but continuously lower for the patch according to the invention.

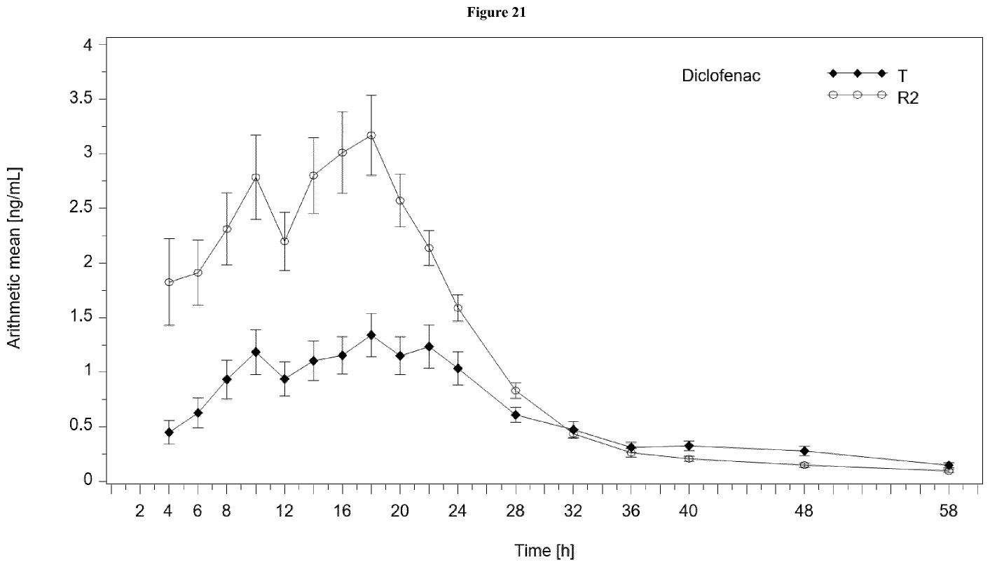

The mean plasma-concentration-time profiles of diclofenac following 18 h application of the patch according to the invention (T) and Flector® (R2)—PK Set are shown in FIG. 21. It can be concluded that the time course of PK time profile for both patches was similar with an overall low exposure but continuously lower for the patch according to the invention.

Summary of mean±standard deviation (coefficient of variation %, n) pharmacokinetic parameters—PK-Set:

| | | tₘₐₓ median | | | | |
|---|---|---|---|---|---|---|
| | | (min-max | Cₘₐₓ | AUC₀₋ₜ | AUC | t_{1/2, z} |
| Compound | Treatment | range) [h] | [ng/mL] | [h*ng/mL] | [h*ng/mL] | [h] |
| Lidocaine | inventive | 16.0 | 24.5 ± 10.0 | 455 ± 175 | 461 ± 173 | 7.7 ± 2.3 |
| | patch | (8.00-22.0) | (41%, 22) | (39%, 22) | (38%, 22) | (30%, 22) |
| | Versatis ® | 14.0 | 34.5 ± 14.7 | 415 ± 235 | 620 ± 233 | 7.0 ± 1.7 |
| | | (10.0-22.0) | (43%, 22) | (38%, 22) | (38%, 22) | (25%, 22) |
| 2,6-Xylidine | inventive | 21.0 | 1.6 ± 0.7 | 39.5 ± 17.7 | 51.7 ± 16.0 | 13.0 ± 2.3 |
| | patch | (14.0-28.0) | (44%, 22) | (45%, 22) | (31%, 15) | (18%, 15) |
| | Versatis ® | 20.0 | 2.2 ± 0.9 | 54.3 ± 21.9 | 68.2 ± 17.8 | 12.6 ± 4.0 |
| | | (14.0-40.0) | (41%, 22) | (40%, 22) | (26%, 17) | (32%, 15) |
| Diclofenac | inventive | 17.9 | 1.6 ± 1.1 | 35.2 ± 20.6 | 40.9 ± 22.5 | 11.1 ± 4.1 |
| | patch | (10.0-24.0) | (64%, 21) | (59%, 21) | (55%, 15) | (37%, 15) |
| | Flector ® | 17.9 | 3.4 ± 1.8 | 63.5 ± 30.7 | 62.1 ± 35.9 | 8.4 ± 2.8 |
| | | (4.0-22.0) | (53%, 22) | (48%, 22) | (58%, 15) | (34%, 15) |

1-68. (canceled)

69. A pharmaceutical patch comprising a Lidocaine constituent and a Diclofenac constituent,
wherein the relative weight ratio of the Lidocaine constituent to the Diclofenac constituent is within the range of from about 7:1 to about 4:1, based on the equivalent weight of the non-salt form of Lidocaine and of Diclofenac,
for topical use in the local treatment or prevention of pain, wherein the pharmaceutical patch is applied and remains applied to an area of the skin of a patient for an application period of more than about 12 hours;
wherein after expiry of the application period, the pharmaceutical patch is removed from the skin, and no other pharmaceutical patch is applied to the area of the skin for an interruption period of at least about 1 hour.

70. The pharmaceutical patch for use according to claim 69, wherein the Diclofenac constituent comprises Diclofenac epolamine.

71. The pharmaceutical patch for use according to claim 69, wherein the Lidocaine constituent comprises Lidocaine in its non-salt form.

72. The pharmaceutical patch for use according to claim 69, wherein the relative weight ratio of the Lidocaine constituent to the Diclofenac constituent is within the range of from about 6.5:1 to about 4.5:1, based on the equivalent weight of the non-salt form of Lidocaine and of Diclofenac.

73. The pharmaceutical patch for use according to claim 69, wherein the total dose of the Lidocaine constituent is within the range of from about 600 mg to about 800 mg, based on the weight of the non-salt form of Lidocaine.

74. The pharmaceutical patch for use according to claim 69, wherein the total dose of the Diclofenac constituent is within the range of from about 85 mg to about 200 mg, based on the weight of the non-salt form of Diclofenac.

75. The pharmaceutical patch for use according to claim 69, which comprises a surface layer, an adhesive layer, and a removable protective layer, wherein the adhesive layer is located between the surface layer and the removable protective layer.

76. The pharmaceutical patch for use according to claim 75, wherein the adhesive layer is a hydrogel or contains a hydrogel comprising at least a portion of the Lidocaine constituent and at least a portion of the Diclofenac constituent.

77. The pharmaceutical patch for use according to claim 69, wherein the pain is low back pain, pain due to osteoarthritis, visceral pain, rheumatoid pain, musculoskeletal pain, joint pain, gout pain, or inflammatory pain.

78. The pharmaceutical patch for use according to claim 69, wherein the application period is less than about 24 hours.

79. The pharmaceutical patch for use according to claim 69, wherein the interruption period is about 6 hours.

80. The pharmaceutical patch for use according to claim 69, wherein the application period lasts about 18 hours and the interruption period lasts about 6 hours.

81. The pharmaceutical patch for use according to claim 69, wherein the pharmaceutical patch is applied to the skin of the breast, or the skin of the knee, or the skin of the elbow, or the skin of the hip, or the skin of the hand, or the skin of the spine, or the skin of the back, particularly of the lower back.

82. A kit comprising a plurality of pharmaceutical patches as defined in claim 69 for use in the local treatment or prevention of pain,
wherein a first pharmaceutical patch is applied and remains applied to an area of the skin of a patient for a first application period of more than about 12 hours, wherein after expiry of the first application period, the first pharmaceutical patch is removed from the skin, and no other pharmaceutical patch is applied to the area of the skin for a first interruption period of at least about 1 hour;
wherein after expiry of the first interruption period, a second pharmaceutical patch is applied and remains applied to the area of the skin of the patient for a second application period of more than about 12 hours, wherein after expiry of the second application period, the second pharmaceutical patch is removed from the skin, and no other pharmaceutical patch is applied to the area of the skin for a second interruption period of at least about 1 hour.

83. The kit according to claim 82, wherein the first application period and the second application period are each about 18 hours, and wherein the first interruption period and the second interruption period are each about 6 hours.
